(19) United States
(12) Patent Application Publication
(10) Pub. No.: US 2006/0128601 A1
(43) Pub. Date: Jun. 15, 2006

(54) Imidazoline quats

(75) Inventors: Abel G. Pereira, Bridgewater, NJ (US); Helena S. Barinova, North Brunswick, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Croda, Inc., Edison, NJ (US)

(21) Appl. No.: 11/338,380

(22) Filed: Jan. 24, 2006

Related U.S. Application Data
(63) Continuation of application No. 10/625,289, filed on Jul. 23, 2003, now abandoned.
Application No. 10/625,289 is a continuation-in-part of application No. 10/339,551, filed on Jan. 9, 2003, now abandoned.
(60) Provisional application No. 60/403,039, filed on Aug. 13, 2002.
(60) Provisional application No. 60/347,170, filed on Jan. 9, 2002.

(51) Int. Cl. A61K 8/00

(52) U.S. Cl. 510/504; 510/130

(57) ABSTRACT

Mixture of dialkyl imidazoline quats with specified substitution content are provided. In one embodiment, at least a portion of the mixture includes at least one dialkyl imidazoline quat having at least one C₁₆-C₃₀ alkyl group; the C₁₆₋₃₀ substitution content of the mixture being from about 10% to about 95% with respect to C₁₀₊ reference substitution range. Methods of making imidazoline quats and quat mixtures, personal care and cosmetic products and formulations that contain the imidazoline quats and quat mixtures, methods of making such personal care and cosmetic products and formulations, and methods of using the such imidazoline quats, quat mixtures, and personal care and cosmetic products and formulations are also provided. Various embodiments are disclosed.

### CROSS-REFERENCE TO RELATED APPLICATIONS

This patent application is a continuation of U.S. application Ser. No. 10/625,289, filed on Jul. 23, 2003, which is a continuation-in-part of U.S. patent application Ser. No. 10/339,551, filed Jan. 9, 2003, which claims the benefit of the filing date of the U.S. Provisional Application No. 60/347,170, filed Jan. 9, 2002, and the filing date of the U.S. Provisional Application No. 60/403,039, filed Aug. 13, 2002, the disclosures of which are hereby incorporated by reference in their entirety.

### BACKGROUND OF THE INVENTION

Compounds containing an imidazoline ring 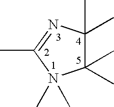
are known in the art. They are disclosed, for example, in U.S. Pat. Nos. 4,851,141, 4,452,732, 4,247,538, 4,206,195, 4,187,289, 4,149,551, and 4,102,795.

U.S. Pat. No. 4,102,795 discloses compositions for softening fabrics or hair that include imidazoline-based quarternary compounds (“quats”) of the formula 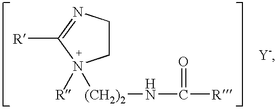
where R′ and R″′ are alkyl groups having 11 to 22 carbon atoms or β-hydroxyalkyl groups having from 13 to 24 carbon atoms; R″ is a lower alkyl group having 1 to 3 carbon atoms, benzyl group, or the group —(C₂H₄O)ₙH, where n is 1 to 3; and Y⁻ is halogen or monoalkyl sulfate. The '795 patent mentions the possibility of mixtures of these compounds, but does not describe any specific mixtures.

U.S. Pat. No. 4,452,732 discloses a shampoo containing several components, including imidazoline-based quaternary compounds of the formula 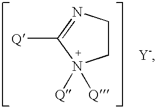
where the groups Q′ and Q″′ is/are a hydrocarbon group(s) containing 16 to 22 carbon atoms, preferably, 16 to 18 carbon atoms; Q″ is C₁-C₄ alkyl or hydroxyalkyl group; and Y⁻ is a compatible anion. The possibility of mixtures is mentioned, again without specificity.

U.S. Pat. No. 4,247,538 discloses compositions for shampooing and conditioning hair that contain several components, including imidazoline-based compounds of the formula 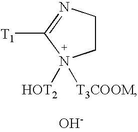
where T₁ is an alkyl group having 12 to 18 carbon atoms; T₂ is an alkylene or hydroxyalkylene group having 1 to 4 carbon atoms; and M is a water-soluble cation. The '538 patent does not disclose mixtures of these compounds.

### SUMMARY OF THE INVENTION

In one aspect, the present invention provides compositions that include a mixture of dialkyl imidazoline quats, where the mixture as a whole has a specified substitution content. Various embodiments are disclosed. In one embodiment, at least a portion of the mixture includes at least one dialkyl imidazoline quat having at least one C₁₆-C₃₀ alkyl group; the C₁₆₋₃₀ substitution content of the mixture being from about 10% to about 95% with respect to C₁₀₊ reference substitution range. In another embodiment, at least a portion of the mixture includes at least one of the dialkyl imidazoline quats has at least one C₂₀-C₃₀ alkyl group; the C₂₀₋₃₀ substitution content of the mixture being from about 10% to about 95% with respect to C₁₀₊ reference substitution range. In another embodiment, at least a portion of the mixture includes at least one of the dialkyl imidazoline quats has at least one C₂₀-C₂₄ alkyl group; the C₂₀₋₂₄ substitution content of the mixture being from about 10% to about 95% with respect to C₁₀₊ reference substitution range.

In another aspect, the invention provides compositions that include a mixture of monoalkyl imidazoline quats, where the mixture as a whole has specified substitution content. Various embodiments are disclosed.

In another aspect, the invention provides imidazoline quat compounds of certain chemical structure. Various embodiments are disclosed.

Methods of making imidazoline quats and quat mixtures, personal care and cosmetic products and formulations that contain the imidazoline quats and quat mixtures, methods of making such personal care and cosmetic products and formulations, and methods of using the such imidazoline quats, quat mixtures, and personal care and cosmetic products and formulations are also provided. Various embodiments of the methods are disclosed.

In another aspect of the present invention there is provided a mixed composition of dialkyl imidazoline quats of the formula (II):

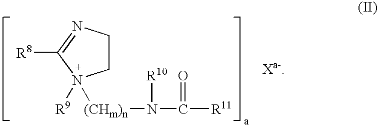

in the above formula X is a salt-forming anion selected from the group consisting of chloride, bromide, iodide, fluoride, sulfate, methyl sulfate, methanebenzylsulfonate, phosphate, nitrite, nitrate, carboxylate, and mixtures thereof and a is the ionic charge of X. n is 1, 2 or 3, m is 1 or 2, and R⁹ is a C₁-C₆ alkyl group or a benzyl group, either of which may be substituted or unsubstituted. R¹⁰ is hydrogen or a C₁-C₆ alkyl group which may saturated or unsatureated and may be hydroxy substituted and R⁸ and R¹¹ may be the same or different and at least 50% of said R⁸ and R¹¹ groups of the mixture are C₁₇-C₃₅ alkyl groups which may be straight, branched or cyclized, may be saturated or unsaturated and may be hydroxy substituted or unsubstituted. Alteratively, for at least about 30% of the dialkyl imidazoline quats both R⁸ and R¹¹ are alkyl groups of at least 19 carbons in length which may be straight, branched or cyclized, may be saturated or unsaturated and may be hydroxy substituted or unsubstituted. Cosmetic and personal care products including these dalkyl imidazoline quat mixtures are also contemplated.

### DESCRIPTION OF THE PREFERRED EMBODIMENTS

For the purposes of the present invention, various terms used herein are defined as follows. A “compound” is a distinct chemical substance having molecules of the same chemical structure. A “compound” is not a mixture of molecules having different chemical structures. A “composition.” may include one compound or a mixture of compounds.

An “alkyl group” is any substituent group that includes a chain of one or more carbon atoms. An alkyl group may terminate in alkyl functionality (e.g., —CH₃) or non-alkyl functionality (e.g., —Br). Likewise, an alkyl group may connect to the rest of the molecule (MOL) through alkyl functionality (e.g., —CH₂— in MOL-CH₂CH₃) or non-alkyl functionality (e.g., —SO₂— in MOL-SO₂C₃H₈). Purely for purposes of illustration, each of the groups —(CH₂)₃—OH, —(CH₂)₄—CH₃, —CH₃, and —C(O)—(CH₂)₅—CH₃, is an alkyl group. An “alkyl radical” is a chain of one or more carbon atoms connected to one another. Purely for purposes of illustration, the alkyl groups —(CH₂)₃—OH, —(CH₂)₄—CH₃, and —C(O)—(CH₂)₅—CH₃ contain alkyl radicals of the structures —(CH₂)₃—, —(CH₂)₄—CH₃, and —(CH₂)₅—CH₃, respectively.

Carbon chains of alkyl groups and alkyl radicals, and alkyl groups and radicals themselves are described as “Cₓ-C_{y}.” An alkyl group containing a Cₓ-C_{y} alkyl radical is referred to as Cₓ-C_{y} alkyl. Such description encompasses carbon chains of every length ranging from x to y carbon atoms, inclusive. For example, the description of an alkyl radical as “C₁₀-C₂₀” encompasses all alternative carbon chains having from 10 to 20 carbon atoms, including carbon chains having 10, 11, 12, 13, 14, 15, 16, 17, 18, 19, and 20 carbon atoms.

Terms such as alkylhydroxy, alkylcarboxy, carboxyalkyl, and the like, are used throughout. Purely for purposes of illustration, an alkylhydroxy group contains a hydroxy group and an alkyl radical, and connects to the rest of the molecule through the alkyl radical; a carboxyalkyl group contains an alkyl radical and a carboxy functionality that connects the carboxy group to the rest of the molecule; an alkylcarboxy group connects to the rest of the molecule through an alkyl radical and terminates in a carboxy functionality. Purely for purposes of illustration, “C₁₀-C₃₀ alkyl” defines a range of alkyl groups containing alkyl radicals having from 10 to 30 carbon atoms and “C₁₀-C₃₀ alkylhydroxy” defines a range of alkyl groups containing a hydroxy group and alkyl radicals having from 10 to 30 carbon atoms.

In the compounds described herein, and consistent with the definitions set forth above, the alkyl groups and alkyl radicals, when present, may be substituted or unsubstituted, straight chain or branched, saturated or unsaturated. The substituents of the alkyl groups and alkyl radicals described herein, when present, may include lower alkyl, which contain alkyl radicals having from 1 to 8 carbon atoms (e.g., methyl, ethyl, n-propyl, i-propyl, and butyl); halogenated lower alkyl, such as trifluoromethyl, perfluoroethyl, chloromethyl, and dichloromethyl; arylalkyl, such as benzyl; alkylaryl, such as p-methylbenzyl; halo, such as fluoro, chloro and bromo; carboxy, such as acetoxy and ethylcarboxy; alkylcarboxy, such as acetoxymethyl and acetoxyethyl; arylacetoxy, such as acetylbenzyl; hydroxy; alkoxy, such as methoxy, ethoxy and propoxy; and alkylhydroxy, such as hydroxymethyl and hydroxyethyl.

“Dialkyl imidazoline quats” are compounds the molecules of which include an imidazoline ring, a quaternary nitrogen atom, and two alkyl groups having 10 or more carbon atoms (two C₁₀₊ alkyl groups). “Monoalkyl imidazoline quats” are compounds the molecules of which include an imidazoline ring, a quaternary nitrogen atom, and one alkyl group having 10 or more carbon atoms (one C₁₀₊ alkyl group).

Mixtures of quat compounds are described herein in terms of their substitution content, which is a characteristic of the quat mixture as a whole. The substitution content of a quat mixture is a ratio, expressed in the percentage terms, of the molar content of the alkyl groups that fall within a specified substitution range to the molar content of the alkyl groups that fall within a broader, reference substitution range. The molar content values for both the specified substitution range and the reference substitution range are measured for the quat mixture as a whole.

The specified substitution ranges are denoted as “C_{x-y}”, indicating a range alkyl groups or alkyl radicals having from x to y carbon atoms. The reference substitution ranges are denoted as “C_{X-Y}” or “C₁₀₊”. “C₁₀₊” indicates a range of alkyl group or alkyl radicals having 10 or more carbon atoms. “C_{X-Y}” indicates a range of alkyl groups or alkyl radicals having from X to Y carbon atoms.

The quat mixtures are described in terms of their “C_{x-y} content” or “C_{x-y} substitution content”. The C₁₀₊ reference range is the default substitution range. Thus, unless specified otherwise, a C_{x-y} substitution content of a quat mixture (abbreviated in the exemplified mixtures throughout as “S_{x-y}”) is the ratio, expressed in the percentage terms, of the molar content of alkyl groups that fall within a C_{x-y} range (“M_{x-y}”) to the molar content of the alkyl groups that fall within the C₁₀₊ range (“M₁₀₊”): S_{x-y}=M_{x-y}/M₁₀₊×100%, where both M_{x-y} and M₁₀₊ are measured for the mixture as a whole. If any reference range other than C₁₀₊ is used to describe a quat mixture (e.g., C_{X-Y} range), the substitution content of the mixture (abbreviated in the exemplified mixtures as “S_{x-y,X-Y}”) and is the ratio of the molar content of alkyl groups that fall within a specified C_{x-y} range (“M_{x-y}”) to the molar content of the alkyl groups that fall within the C_{X-Y} reference range (“M_{X-Y}”): S_{x-y/X-Y}=M_{x-y}/M_{x-y}×100%.

To illustrate, consider the mixture M1 that contains a single molecule of dialkyl quat A1 and a single molecule of different dialkyl imidazoline quat A2. By definition, each of dialkyl quats A1 and A2 has two C₁₀₊ alkyl groups. Suppose, the molecule of quat Al has one C₂₀ alkyl group and one C₁₂ alkyl group, and the molecule of quat A2 has two C₂₀ alkyl groups. Suppose also, the mixture M1 is to be characterized in terms of its C₁₆-₃₀ substitution content. (S₁₆₋₃₀(M1)), e.g., the narrower, specified range is C₁₆₋₃₀ and the broader, reference range is C₁₀₊.

The C₁₆₋₃₀ substitution content of the mixture M1 can be calculated as: S₁₆₋₃₀(M1)=M₁₆₋₃₀(M1)/M₁₀₊(M1)×100%, where M₁₆₋₃₀ is the C₁₆₋₃₀ molar content of the mixture M1 and M₁₀₊(M1) is the C₁₀₊ molar content of the mixture M1. Since the molar concentrations of quats A1 and A2 in the mixture M1 are identical (one molecule each), the relative molar concentration may be disregarded, and the absolute numbers of alkyl groups falling within each range may be used instead. Thus, the C₁₆₋₃₀ substitution content of the mixture M1 as a whole may be calculated as: S₁₆₋₃₀(M1)=N₁₆₋₃₀(M1)/N₁₀₊(M1)×100%, where N₁₆₋₃₀(M1) is the number of alkyl groups in the mixture M1 that fall within the C₁₆₋₃₀ range and N₁₀₊(M1) is the number of alkyl groups in the mixture M1 falling within the C₁₀₊ range.

The first step is to calculate the values of N₁₆₋₃₀ and N₁₀₊ for the mixture. To determine N₁₆₋₃₀ and N₁₀₊, a substituent group is counted every time it falls within the C₁₀₊ and C₁₆₋₃₀ ranges, respectively, for all molecules in the mixture. The same group may be counted more than once. The C₂₀ group falls within both the C₁₆₋₃₀ range and the C₁₀₊ range and thus should be counted in calculating both N₁₆₋₃₀(M1) and N₁₀₊(M1), while the C₁₂ group fall only within the C₁₀₊ range and therefore should be counted only in calculating N₁₀₊(M1). Performing the calculation for the mixture M1 as a whole, N₁₆₋₃₀ is 3 (one C₂₀ group of quat A1 and two C₂₀ group of the quat A2) and N₁₀₊ is 4 (all four groups are in the C₁₀₊ range). Therefore, S₁₆₋₃₀ for the mixture M1 is 75% (¾×100%).

For more complex quat mixtures, molar concentrations of quats in the mixture are taken into account. A non-limiting example illustrates calculation of substitution content for mixture M2 of dialkyl quats A3, A4, and A5. The mixture M2 is characterized in terms of its C₂₀₋₃₀ substitution content (the specified substitution range is C₂₀₋₃₀ and the reference substitution range is C₁₀₊):

TABLE 1*

| | | | IV | | |
|---|---|---|---|---|---|
| | II | III | (M₀// | V | VI |
| I | (N₂₀₋₃₀) | (P₂₀₋₃₀) | M₁₀₊) | (M₂₀₋₃₀) | (S₂₀₋₃₀) |
| Quat | Number of | C₂₀₋₃₀ | Moles | C₂₀₋₃₀ molar | C₂₀₋₃₀ |
| | alkyl groups | molecular | | contribution | substitution |
| | in the quat | content | | of each quat | content of |
| | molecule | for each | | ((III) × ((IV)) | the mixture |
| | falling in | quat | | and C₂₀₋₃₀ | |
| | the C₂₀₋₃₀ | ((II)/2) | | molar | |
| | range | | | content of the | |
| | | | | mixture | |
| A3 | 1 (one) | 0.5 | 2 | 1 (0.5 × 2) | |
| A4 | 2 (two) | 1 | 0.75 | 0.75 (1 × 0.75) | |
| A5 | 0 (none) | 0 | 0.75 | 0 (0 × 0.75) | |
| M2 | | | 3.5 | 1.75 | 50% |
| | | | | | (1.75/3.5) |

*Column (I) identifies quat components of the mixture M2. Column (II) indicates how many alkyl groups of each quat molecule fall within the C₂₀₋₃₀ range (note that dialkyl quats have two groups in the C₁₀₊ range (N₁₀₊ is 2)). Column (IV) provides molar amounts of each quat in the mixture M2 and the total number of quat moles in the mixture M2. Columns (III), (V) and (VI) are explained below.

To calculate the C₂₀₋₃₀ substitution content (S₂₀₋₃₀) of the mixture M2, the first step is to determine the C₂₀₋₃₀ content the molecule of each quat based on its chemical structure. Such substitution content is referred to as C₂₀₋₃₀ “molecular content” and denoted as “P₂₀₋₃₀”. The C₂₀₋₃₀ molecular content of a quat is determined by dividing the number of C₂₀-C₃₀ alkyl groups (N₂₀₋₃₀) by the number of C₁₀₊ alkyl groups (N₁₀₊) in molecule of the quat: P₂₀₋₃₀=N₂₀₋₃₀/N₁₀₊.

In the example, the nature of substitution for quats in the mixture is provided in column (II). The molecule of quat A3 has 1 (one) group that falls in the C₂₀₋₃₀ range. The number of groups in the C₁₀₊ range is 2 (two) for all dialkyl quats. Thus, the C₂₀₋₃₀ molecular content of quat A3 (P₂₀₋₃₀ (A3)) is ½=0.5. The C₂₀₋₃₀ molecular content values for quats A3, A4, and A5 are calculated in the same manner by dividing the values in column (II) by 2, and are shown in column (III).

Next, the C₂₀₋₃₀ molar contribution of each quat component of the mixture (M₂₀₋₃₀) is calculated. For this purpose, each quat's C₂₀₋₃₀ molecular content (P₂₀₋₃₀, column (III)) is multiplied by the number of moles of the corresponding quat in the mixture (M₀, column (IV)): M₂₀₋₃₀=P₂₀₋₃₀×M₀. The results of the calculations are shown in column (V). In effect, the product of the multiplication is the molar amount of C₂₀₋₃₀ alkyl groups contributed by each quat component of the mixture M2.

The C₂₀₋₃₀ molar content of the mixture M2 as a whole (M₂₀₋₃₀(M2)) is the sum of the C₂₀₋₃₀ molar contributions of individual quats: M₂₀₋₃₀(M2)=M₂₀₋₃₀(A3)+M₂₀-₃0(A4)+M₂₀₋₃₀(A5) Referring to column (V), quat A3 contributes 1 mole of C₂₀-C₃₀ groups (M₂₀₋₃₀(A3)=1), quat A4 contributes 0.75 moles (M₂₀₋₃₀(A4)=0.75), and quat A5 contributes 0 moles of C₂₀-C₃₀ groups (M₂₀₋₃₀(A5)=0). Therefore, the C₂₀₋₃₀ molar content of the mixture M2 is 1.75 (M₂₀₋₃₀(M2)=1+0.75+0).

The sum of the C₁₀₊ molar contributions of individual quats is the C₁₀₊ molar content of the mixture M2 as a whole (M₁₀₊(M2)): M₁₀₊(M2)=M₁₀₊(A3)+M₁₀₊(A4)+M₁₀₊(A5). Since all dialkyl quats have two alkyl groups in the C₁₀₊ range, the C₁₀₊ molar content (M₁₀₊) of a dialkyl quat component is identical to the number of moles of the quat component (M₀). Referring to column (IV), quat A3 contributes 2 mole of C₁₀₊ groups (M₀₊(A3)=2), quat A4 contributes 0.75 moles (M₀₊(A4)=0.75), and quat A5 contributes 0.75 moles of C₁₀₊ groups (M₀₊(A5)=0.75). Therefore, the C₁₀₊ molar content of the mixture M2 is 3.5 (M₁₀₊(M2)=2+0.75+0.75). Finally, the C₂₀₋₃₀ substitution content of the mixture M2. can be calculated: S₂₀₋₃₀(M2)=M₂₀₋₃₀(M2)/M₁₀₊(M2)=1.75/3.5=50% (column (VI)).

Another non-limiting example illustrates calculation of the substitution content for mixtures of monoalkyl quats. Table 2 shows mixture M3 of monoalkyl quats B1, B2, and B3. The mixture M3 is characterized in terms of its C₂₀₋₂₄ substitution content (the specified substitution range is C₂₀₋₂₄ and the reference substitution range is C₁₀₊).

TABLE 2

| | | | IV | | |
|---|---|---|---|---|---|
| | II | III | (M₀// | V | VI |
| I | (N₂₀₋₂₄) | (P₂₀₋₂₄) | M₁₀₊) | (M₂₀₋₂₄) | (S₂₀₋₂₄) |
| Quat | Number of | C₂₀₋₂₄ | Moles | C₂₀₋₂₄ molar | C₂₀₋₂₄ |
| | alkyl groups | molecular | | contribution | substitution |
| | (the C₂₀₋₂₄ | content | | of each quat | content of |
| | range) that | for each | | (III × IV) and | the mixture |
| | fall in the | quat | | C₂₀₋₂₄ molar | (V/IV) |
| | quat | (II/1) | | content the | |
| | molecule | | | mixture | |
| B1 | 1 | 1 | 1.5 | 1.5 (1 × 1.5) | |
| B2 | 1 | 1 | 1 | 1 (1 × 1) | |
| B3 | 0 | 0 | 2.5 | 0 (0 × 2.5) | |
| M3 | | | 5 | 2.5 | 2.5/5 × |
| | | | | | 100% = 50% |

The C₂₀₋₂₄ substitution content of the mixture M3 (S₂₀₋₂₄ (M3)) is calculated as follows similarly to the calculations described in reference to the mixture of Table 1:

1. Determine the C₂₀₋₂₄ molecular content for each monoalkyl quat: P₂₀₋₂₄=N₂₀₋₂₄/N₁₀₊. Since N₁₀₊ is 1 (one) for all monoalkyl quats, the C₂₀₋₂₄ molecular content for each quat is calculated as: P₂₀₋₂₄=N₂₀₋₂₄/1. The results are shown in column (III). It is evident that P_{x-y}=N_{x-y} for monoalkyl quats in general. Thus, N_{x-y} may be used instead of P_{x-y} and column (III) is omitted in later examples of monoalkyl quat mixtures.

2. Determine the C₂₀₋₂₄ molar contributions of quat components by multiplying the number of C₂₀-C₂₄ groups by molar amount for each quat component: M₂₀₋₂₄=N₂₀₋₂₄×M₀. The N₂₀₋₂₄ values in column (II) are multiplied by the corresponding M₀ values in column (IV). The results of the calculations are in column (V).

3. Determine the C₂₀₋₂₄ molar content of the mixture M3 by adding the C₂₀₋₂₄ molar contributions of the quat components: M₂₀₋₂₄(M3)=M₂₀₋₂₄(B1)+M₂₀₋₂₄(B2)+M₂₀₋₂₄(B3). The M₂₀₋₂₄ values for each quat component in column (V) are added. The calculated C₂₀₋₂₄ molar content of the mixture M3 is also shown in column (V).

4. Determine the C₁₀₊ molar content of the mixture M3 by adding the C₁₀₊ molar contributions of the quat components. Since all monoalkyl quats have one alkyl group in the C₁₀₊ range, molar amounts of the quats are used: M₁₀₊(M3)=M₀(B1)+M₀(B2)+M₀(B3). The M₀ values for each quat component in column (IV) are added. The calculated C₁₀₊ molar content of the mixture M3 is shown in column (IV).

5. Determine the C₂₀₋₂₄ substitution content of the mixture M3 by dividing the C₂₀₋₂₄ molar content of the mixture M3 by the C₁₀₊ molar content of the quats in the mixture: S₂₀₋₂₄(M3)=M₂₀₋₂₄(M3)/M₁₀₊(M3). The calculated C₂₀₋₂₄ substitution content of the mixture M3 is shown in column (VI).

The above definitions and calculation methodologies are used throughout to describe various aspects and embodiments of the invention.

In accordance with one aspect, the invention provides compositions that include mixtures of at least two different dialkyl imidazoline quats; the mixture having specified substitution content. In addition to dialkyl imidazoline quats, the compositions of this aspect of the invention may include other quaternary and non-quaternary compounds. If desired, monoalkyl imidazoline quats and/or other quaternary compounds are substantially excluded from the composition.

In one embodiment, the mixture includes at least one dialkyl imidazoline quat with one or both alkyl groups being C₁₆₋₃₀ alkyl groups. Thus, at least a portion of the mixture has C₁₆-C₃₀ alkyl groups, providing the mixture as a whole with C₁₆₋₃₀ substitution content greater than zero. In this embodiment, the C₁₆₋₃₀ substitution content of the mixture is from about 10% to about 95%, more preferably, from about 15% to about 80%, yet more preferably, from about 20% to about 70%, yet more preferably, from about 35% to about 60%.

In another embodiment, the mixture includes at least one dialkyl imidazoline quat with one or both alkyl groups being C₂₀-C₃₀ alkyl groups. Thus, at least a portion of the mixture has C₂₀-C₃₀ alkyl groups, providing the mixture as a whole with C₂₀-₃₀ substitution content greater than zero. In this embodiment, the C₂₀₋₃₀ substitution content of the mixture is from about 10% to about 95%, more preferably, from about 15% to about 80%, yet more preferably, from about 20% to about 70%, yet more preferably, from about 35% to about 60%.

In yet one embodiment, the mixture includes at least one dialkyl imidazoline quat with one or both alkyl groups being C₂₀-C₂₄ alkyl groups. Thus, at least a portion of the mixture has C₂₀-C₂₄ alkyl groups, providing the mixture as a whole with C₂₀₋₂₄ substitution content greater than zero. In this embodiment, the C₂₀₋₂₄ substitution content of the mixture is from about 10% to about 95%, more preferably, from about 15% to about 80%, yet more preferably, from about 20% to about 70%, yet more preferably, from about 35% to about 60%.

Dialkyl imidazoline quats in the mixtures may be selected from compounds of the formula (I): 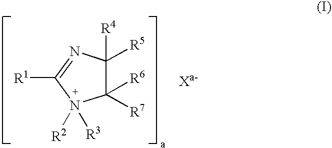
where X is a salt-forming anion, such as chloride, bromide, iodide, fluoride, sulfate, methyl sulfate, methanebenzylsulfonate, phosphate, nitrite, nitrate, carboxylate, or a mixture thereof, preferably, chloride or methyl sulfate; a is the ionic charge of X;

R¹, R², and R³ are independently hydrogen, C₁-C₃₀ alkyl, C₁-C₃₀. alkylhydroxy, C₁-C₃₀ alkyl amido R_{(C1-C6)} wherein R_{(C1-C6)} is a C₁-C₆ alkylene or benzyl, C₁-C₃₀ alkylaryl amido R_{(C1-C6)} or C₁-C₃₀ alkylhydroxy amido R_{(C1-C6)};

two of R¹, R², and R³ are independently C₁₀-C₃₀ alkyl, C₁₀-C₃₀ alkylhydroxy, C₁₀-C₃₀ alkyl amido R_{(C1-C6)}, C₁₀-C₃₀ alkylaryl amido R_{(C1-C6)}, or C₁₀-C₃₀ alkylhydroxy amido R_{(C1-C6)}, and the remaining one of R¹, R² and R³ is hydrogen, C₁-C₈ alkyl, C₁-C₈ alkylhydroxy, C₁-C₈ alkyl amido R_{(C1-C6)}, C₁-C₈ alkylaryl amido R_{(C1-C6)}, or C₁-C₈ alkylhydroxy amido R_{(C1-C6)};

R⁴, R⁵, R⁶ and R⁷ same or different, are independently hydrogen, alkyl, arylalkyl, alkylaryl, halogen, including bromo and chloro, acetoxy, alkylacetoxy, arylacetoxy, carboxy, alkylcarboxy, hydroxy, or alkoxyhydroxy, preferably, R⁴, R⁵, R⁶, and R⁷, same or different, are hydrogen or C₁-C₈ alkyl.

More preferably, the dialkyl imidazoline quats in the mixtures are selected from the compounds of the formula (I) in which R¹ is C₁₀-C₃₀ alkyl or C₁₀-C₃₀ alkylhydroxy, R² is C₁-C₆ alkyl, R³ is C₁₀-C₃₀ alkyl amido R_{(C1-C6)} or C₁₀-C₃₀ alkylhydroxy amido R_{(C1-C6)} and R⁴, R⁵, R⁶, and R⁷ are independently hydrogen or C₁-C₈ alkyl. Yet more preferably, R¹ is C₁₀-C₃₀ alkyl, R² is methyl, R³ is C₁₀-C₃₀ alkyl amido C₁-C₃ alkylene or C₁₀-C₃₀ alkylhydroxy amido C₁-C₃ alkylene, R⁴, R⁵, R⁶, and R⁷ are hydrogen, and X is chloride or methyl sulfate.

The portion providing the mixture with substitution content greater than C₁₀₊, such as the C₁₆₋₃₀, C₂₀₋₃₀ or C₂₀₋₂₄ substitution contents, may contain compounds of the formula (I) in which R¹ is C₁₆-C₃₀ alkyl or C₁₆-C₃₀ alkylhydroxy, and R³ is C₁₀-C₃₀. alkyl amido R_{(C1-C6)}; or compounds of the formula (I) in which R¹ is C₁₀-C₃₀ alkyl or C₁₀-C₃₀ alkylhydroxy, and R³ is C₁₆-C₃₀ alkyl amido R_{(C1-C6)}; or compounds of the formula (I) in which both R¹ and R³ are in the C₁₆₋₃₀ substitution range. The portion of the mixture may have the same relative variations of the content of the portion for C₁₆-C₃₀ and C₂₀-C₂₄ substituents.

Non-limiting examples of dialkyl quats of the formula (I) are quats (1), (2), (3), and (4):

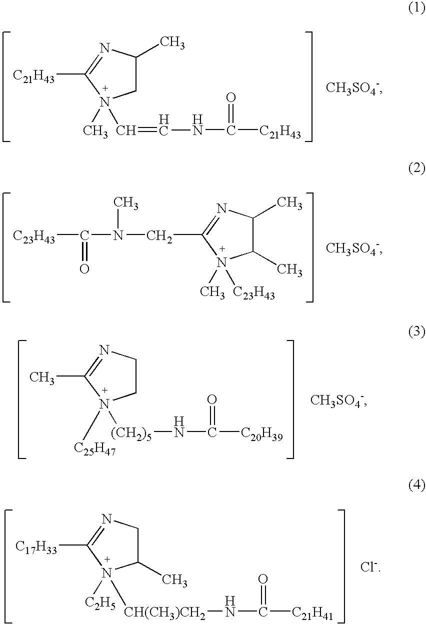

In another non-limiting example, Table 3 shows mixture M4 of dialkyl quats (1), (2), (3), and (4) and the calculation of the mixture's substitution content. The mixture M4 is characterized in terms of its C₂₀₋₃₀ substitution content (the specified substitution range is C₂₀₋₃₀ and the reference substitution range is C₁₀₊).

TABLE 3*

| | | III | | V | VI |
|---|---|---|---|---|---|
| | II | (P₂₀₋₃₀ = | IV | (M₂₀₋₃₀ = | (S₂₀₋₃₀ = V/ |
| I | (N₂₀₋₃₀) | II/2) | (M₀//M₁₀₊) | III × IV) | IV) |
| 1 | 1 | 0.5 | 1 | 0.5 (0.5 × 1) | |
| 2 | 2 | 1 | 2 | 2 (1 × 2) | |
| 3 | 2 | 1 | 2 | 2 (1 × 2) | |
| 4 | 1 | 0.5 | 3 | 1.5 (0.5 × 3) | |
| M4 | | | 8 | 6 | 75% |
| | | | | | (6/8 × 100%) |

*Explanations for calculating the C₂₀₋₃₀ content are provided with reference to Table 1.

The invention also provides compositions that include a mixture of dialkyl imidazoline quats of the formula (II):

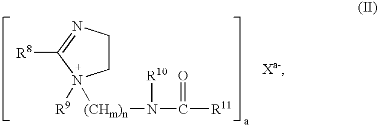

where X and a are defined above with reference to the formula (I);

n varies from 1 to 3, preferably, n is 2;

m is 1 or 2, preferably, m is 2;

R⁸ and R¹¹, which may be the same or different, are C₁₆-C₃₀ alkyl;

R⁹ is hydrogen or C₁-C₃ alkyl, preferably, methyl;

R¹⁰ is hydrogen, alkyl, arylalkyl, alkylaryl, halogen, including bromo and chloro, acetoxy, alkylacetoxy, arylacetoxy, carboxy, alkylcarboxy, hydroxy, or alkoxyhydroxy, preferably, hydrogen or lower alkyl, more preferably, hydrogen.

In addition to dialkyl imidazoline quats of the formula (II), the compositions of this embodiment may include other quaternary and non-quaternary compounds. If desired, monoalkyl imidazoline quats and/or other quaternary compounds may be substantially excluded from the composition.

In one embodiment, at least a portion of the mixture includes at least one quat of the formula (II) in which one or both of R⁸ and R¹¹ is/are C₁₆-C₂₄ alkyl group(s), and the C₁₆₋₂₄ substitution content of the mixture with respect to the C₁₆₋₃₀ reference substitution range varies from about 10% to about 95%, more preferably, from about 15% to about 80%, more preferably, from about 20% to about 80%, yet more preferably, from about 35% to about 60%.

In another embodiment, at least a portion of the mixture includes at least one quat of the formula (II) in which one or both of R⁸ and R¹¹ is/are C₂₀-C₂₄ alkyl group(s) and the C₂₀₋₂₄ substitution content of the mixture with respect to the C₁₆₋₃₀ reference substitution range varies from about 10% to about 95%, more preferably, from about 15% to about 80%, yet more preferably, from about 20% to about 70%, yet more preferably, from about 35% to about 60%.

Non-limiting examples of dialkyl imidazoline quats of the formula (II) are quats (5.), (6), (7), and (8):

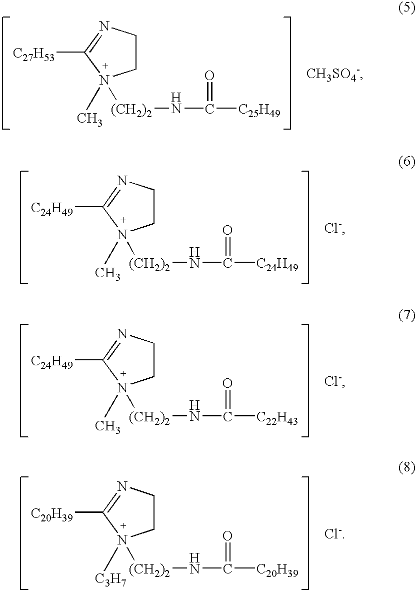

Other non-limiting examples of the quats of the formula (II) are quats (9)-(16) shown in Table 4:

TABLE 4*

| Compound | R⁸ | R¹¹ |
|---|---|---|
| 9 | C₁₈H₃₅ | C₁₈H₃₅ |
| 10 | C₂₁H₄₀ | C₂₁H₄₀ |
| 11 | C₂₂H₄₀ | C₂₂H₄₀ |
| 12 | C₁₆H₂₉ | C₁₈H₃₅ |
| 13 | C₂₃H₄₀ | C₂₃H₄ |
| 14 | C₁₈H₃₅ | C₂₂H₄₁ |
| 15 | C₁₈H₃₃ | C₁₈H₃₃ |
| 16 | C₂₂H₄₀ | C₂₂H₄₀ |

*R⁹ is methyl, R¹⁰ is hydrogen, m is 2, and n is 2.

In a non-limiting example, Table 5 shows mixture M5 of dialkyl quats (9), (10), and (11) and the calculation of the mixture's substitution content. The mixture M5 is characterized in terms of its C_{20-24/16-30} substitution content (the specified range is C₂₀₋₂₄ and the reference range is C₁₆₋₃₀). The C₁₆₋₃₀ range is used as the reference range, instead of the default C₁₀₊ reference range, since R⁸ and R¹¹ are C₁₆-C₃₀ alkyl groups. The combined molar content of R⁸ and R¹¹ groups in the mixture M5 is used in the calculations:

TABLE 5

| | | III | IV | V | VI |
|---|---|---|---|---|---|
| I | II | (P₂₀₋₂₄ = | (M₀// | (M₂₀₋₂₄ = | (S_{20-24/16-30} = |
| (Quat) | (N₂₀₋₂₄) | II/2) | M₁₀₊) | III × IV) | V/IV) |
| 9 | 0 | 0 | 1 | 0 (0 × 1) | |
| 10 | 2 | 1 | 0.75 | 0.75 (0.75 × 1) | |
| 11 | 2 | 1 | 0.25 | 0.25 (0.25 × 1) | |
| M5 | | | 2 | 1 | 50% |
| | | | | | (1/2 × 100%) |

Other non-limiting examples of quat mixtures and calculations of their C₂₀₋₂₄ content are shown in Tables 6 and 7:

TABLE 6

| | | III | IV | V | VI |
|---|---|---|---|---|---|
| I | II | (P₂₀₋₂₄ = | (M₀// | (M₂₀₋₂₄ = | (S_{20-24/16-30} = V/ |
| (Quat) | (N₂₀₋₂₄) | II/2) | M₁₀₊) | III × IV) | IV) |
| 12 | 0 | 0 | 1 | 0 (0 × 1) | |
| 13 | 2 | 1 | 2.5 | 2.5 (1 × 2.5) | 71.4% |
| M6 | | | 3.5 | 2.5 | (2.5/3.5 × 100%) |

TABLE 7

| | | III | IV | V | VI |
|---|---|---|---|---|---|
| I | II | (P₂₀₋₂₄ = | (M₀// | (M₂₀₋₂₄ = | (S_{20-24/16-30} = V/ |
| (Quat) | (N₂₀₋₂₄) | II/2) | M₁₀₊) | III × IV) | IV) |
| 14 | 1 | 0.5 | 2 | 1 (0.5 × 2) | |
| 15 | 0 | 0 | 1 | 0 (0 × 1) | |
| 16 | 2 | 1 | 1 | 1 (1 × 1) | |
| M7 | | | 4 | 2 | 50% |
| | | | | | (2/4 × 100%) |

In another aspect, the invention provides a composition that include a dialkyl imidazoline quat of the formula (IA):

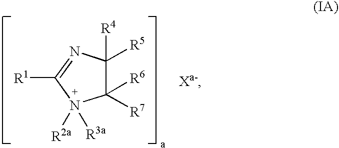

where X is a salt-forming anion, such as chloride, bromide, iodide, fluoride, sulfate, methyl sulfate, methanebenzylsulfonate, phosphate, nitrite, nitrate, carboxylate, and mixtures thereof;

a is the ionic charge of X;

R^{1a}, R^{2a}, and R^{3a} are independently hydrogen, C₁-C₃₆ alkyl, C₁-C₃₆ alkylhydroxy, C₁-C₃₆ alkyl amido R_{(C1-C6)}, C₁-C₃₆ alkylaryl amido R_{(C1-C6)} or C₁-C₃₆ alkylhydroxy amido R_{(C1-C6)}, R_{(C1-C6)} being C₁-C₆ alkylene or benzyl;

two of R^{1a}, R^{2a}, and R^{3a} are independently C₁₀-C₃₆ alkyl, C₁₀-C₃₆ alkylhydroxy, C₁₀-C₃₆ alkyl amido R_{(C1-C6)}, C₁₀-C₃₆ alkylaryl amido R_{(C1-C6)} or C₁₀-C₃₆ alkylhydroxy amido R_{(C1-C6)};

the remaining one of R^{1a}, R^{2a} and R^{3a} is hydrogen, C₁-C₈ alkyl, C₁-C₈ alkylhydroxy, C₁-C₈ alkyl amido R_{(C1-C6)}; C₁-C₈ alkylaryl amido R_{(C1-C6)} or C₁-C₈ alkylhydroxy amido R_{(C1-C6)}; R⁴, R⁵, R⁶, and R⁷, same or different, are independently hydrogen, alkyl, arylalkyl, alkylaryl, fluoro, bromo, chloro, iodo, acetoxy, alkylacetoxy, arylacetoxy, carboxy, alkylcarboxy, hydroxy or alkoxyhydroxy;

with the proviso that the dialkyl imidazoline quat does not have the formula

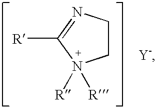

where R′ is C₁₁-C₂₂ alkyl or C₁₃-C₂₄ β-alkyl hydroxy; R″ is C₁-C₆ alkyl; and R″′ is C₁₂-C₂₀ alkyl or C₁₁-C₂₂ alkyl amido C₁-C₃ alkylene.

In one embodiment of this aspect, the dialkyl imidazoline quat is a compound of the formula (III):

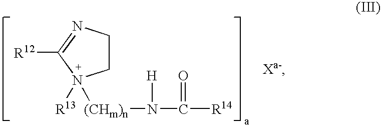

where n varies from 1 to 3, preferably, n is 2;

m is 1 or 2, preferably, m is 2;

R¹² and R¹⁴, same or different, are C₂₄-C₃₀ alkyl; and

R¹³ is hydrogen or C₁-C₃ alkyl.

In yet another aspect, the invention provides compositions that include mixtures of at least two monoalkyl imidazoline quats with specified substitution content. In addition to monoalkyl, imidazoline quats, the compositions may include other quaternary and non-quaternary compounds. If desired, dialkyl imidazoline quats and/or other quaternary compounds may be substantially excluded from the composition.

In one embodiment, the mixture contains monoalkyl imidazoline quats, at least one of which has a C₁₆-C₃₀ alkyl group; the C₁₆₋₃₀ substitution content of the mixture being from about 10% to about 95%, preferably, from about 15% to about 85%, more preferably, from about 20% to about 80%, yet more preferably, from about 25% to about 75%.

In another embodiment, the mixture contains monoalkyl imidazoline quats at least one of which has a C₁₈-C₂₆ alkyl group; the C₁₈₋₂₆ substitution content of the mixture being from about 10% to about 95%, preferably, from about 15% to about 85%, more preferably, from about 20% to about 80%, yet more preferably, from about 25% to about 75%.

In yet another embodiment, the mixture contains monoalkyl imidazoline quats at least one of which has a C₂₀-C₂₄ alkyl group; the C₂₀₋₂₄ substitution content of the mixture being from about 10% to about 90%, preferably, from about 15% to about 85%, more preferably, from about 20% to about 80%, yet more preferably, from about 25% to about 75%.

Monoalkyl quats in the mixtures may have the formula (IV):

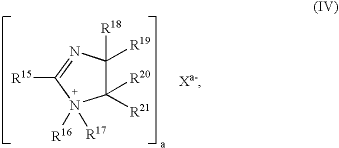

where X and a are defined in reference to formula (I);

R¹⁵, R¹⁶ and R¹⁷ are independently hydrogen, C₁-C₃₀ alkyl, C₁-C₃₀ alkylhydroxy, C₁-C₃₀ alkyl amido R_{(C1-C6)}, C₁-C₃₀ alkylaryl amido R_{(C1-C6)}, or C₁-C₃₀ alkylhydroxy amido R_{(C1-C6)}, wherein R_{(C1-C6)} is a C₁-C₆ alkylene or benzyl;

one of R¹⁵, R¹⁶ and R¹⁷ is C₁-C₃₀ alkyl, C₁₀-C₃₀ alkylhydroxy, C₁₀-C₃₀ alkyl amido R_{(C1-C6)}, C₁₀-C₃₀ alkylaryl amido R_{(C1-C6)} or C₁₀-C₃₀ alkylhydroxy amido R_{(C1-C6)};

the remaining two of R¹⁵, R¹⁶ and R¹⁷ are independently hydrogen, C₁-C₈ alkyl, C₁-C₈ alkylhydroxy, C₁-C₈ alkyl amido R_{(C1-C6)}, C₁-C₈ alkylaryl amido R_{(C1-C6)}, or C₁-C₈ alkylhydroxy amido R_{(C1-C6)};

R¹⁸, R¹⁹, R²⁰, and R²¹, same or different, are independently hydrogen, alkyl, arylalkyl, alkylaryl, halogen; including bromo and chloro, acetoxy, alkylacetoxy, arylacetoxy, carboxy, alkylcarboxy, hydroxy, or alkoxyhydroxy; preferably, R¹⁸, R¹⁹, R²⁰, and R²¹, same or different, are hydrogen or C₁-C₈ alkyl.

More preferably, R¹⁵ is C₁₀-C₃₀ alkyl or alkylhydroxy, yet more preferably, C₁₄-C₃₀ alkyl or alkylhydroxy, yet more preferably, R¹⁵ is C₁₆-C₃₀ alkyl or or alkylhydroxy, yet more preferably, R¹⁵ is C₂₀-C₃₀ alkyl or alkylhydroxy;

R¹⁶ is C₁-C₆ alkyl, yet more preferably, C₁-C₃ alkyl, yet more preferably methyl;

R¹⁷ is C₁-C₈ alkyl, C₁-C₈ alkylhydroxy, C₁-C₈ alkyl amido R_{(C1-C6)} or C₁-C₈ alkylhydroxy amido R_{(C1-C6)} more preferably, C₁-C₈ alkyl amido C₁-C₃ alkylene or C₁-C₈ alkylhydroxy.

Non-limiting examples of monoalkyl quats of the formula (IV) are quats (17), (18), and (19):

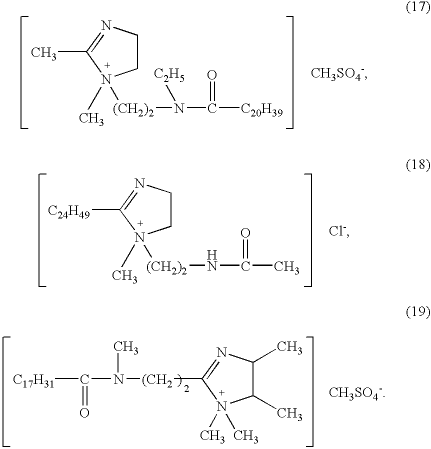

More preferably, monoalkyl quats in the mixtures have the formula (V) 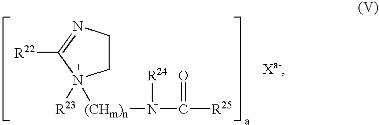
or formula (VI)

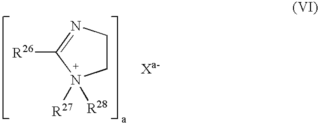

where X and a are as defined above with reference to the formula (I);

R²² and R²⁶ are independently C₁₆-C₃₀ alkyl;

R²³ and R²⁷ are each hydrogen or C₁-C₃ alkyl;

R²⁴ is hydrogen, alkyl, arylalkyl, alkylaryl, halogen, including bromo and chloro, acetoxy, alkylacetoxy, arylacetoxy, carboxy, alkylcarboxy, hydroxy, or alkoxyhydroxy;

R²⁵ and R²⁸ are each C₁-C₈ alkyl or C₁-C₈ alkylhydroxy;

n varies from 1 to 3; and

m is 1 or 2.

Yet more preferably, R²⁴ is hydrogen or lower alkyl, yet more preferably, hydrogen; n is 2; and m is 2.

Non-limiting examples of monoalkyl quats of the formula (V) are quats (20) and (21):

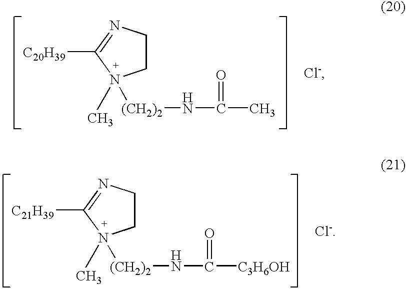

Non-limiting examples of monoalkyl quats of the formula (VI) are quats (22) and (23):

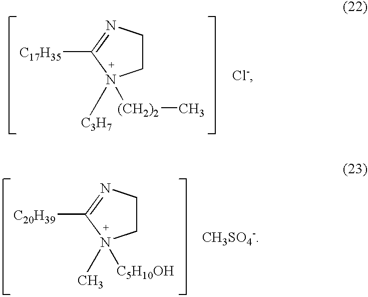

Other non-limiting examples of monoalkyl quats of the formula (V) are quats (24)-(28) shown in Table 8:

TABLE 8*

| Quat | R²² |
|---|---|
| 24 | C₁₈H₃₅ |
| 25 | C₂₁H₄₀ |
| 26 | C₂₂H₄₁ |
| 27 | C₁₈H₃₃ |
| 28 | C₂₂H₄₀ |

*R²³ is CH_{3;} R²⁴ is hydrogen, R²⁵ is CH₃

In non-limiting examples, Tables 9 and 10 show mixtures M8 and M9, respectively, of quats (24)-(28) and the calculations of their substitution content. Both mixtures are characterized in terms of their C₂₀₋₂₄ content (the specified range is C₂₀₋₂₄ and the reference range is C₁₀₊).

TABLE 9*

| I | II | IV | V | VI |
|---|---|---|---|---|
| (Quat) | (N₂₀₋₂₄) | (M₀//M₁₀₊) | (M₂₀₋₂₀ = II × IV) | (S₂₀₋₂₄ = V/IV) |
| 24 | 0 | 1 | 0 (0 × 1) | |
| 25 | 1 | 1.5 | 1.5 (1 × 1.5) | |
| M8 | | 2.5 | 1.5 | 60% |
| | | | | (1.5/2.5 × 100%) |

*N₁₀₊ is 1; column (III) is omitted

TABLE 10*

| I | II | IV | V | VI |
|---|---|---|---|---|
| (Quat) | (N₂₀₋₄ | (M₀//M₁₀₊) | (M₂₀₋₂₄ = II × IV) | (S₂₀₋₂₄ = V/IV) |
| 26 | 1 | 2 | 2 (1 × 2) | |
| 27 | 0 | 1 | 0 (0 × 1) | |
| 28 | 1 | 1 | 1 (1 × 1) | |
| M9 | | 4 | 3 | 75% |
| | | | | (¾) × 100% |

Monoalkyl imidazoline quats and dialkyl imidazoline quats may be prepared in a number of ways, including methods known to those skilled in the art. One of suitable preparation methods is described in U.S. Pat. No. 4,855,440, incorporated herein by reference in its entirety. A possible synthetic route involves a reaction of a carboxylic acid, anhydride, or natural or synthetic oil, with a desired dialkyltriamine (for dialkyl quats preparation) or N-alkyl-N-alkylamino-diamine (for monoalkyl quats preparation), followed by quaternization of the resulting imidazoline intermediate.

Reaction Scheme 1 shows an example of the synthetic route for preparation of certain dialkyl imidazoline quats, specifically, 1-methyl-1-(alkyl-acylamido-) ethyl)-2-alkyl immidazolinium methyl sulfates, via a reaction between one mole of diethylene triamine and two moles of a fatty carboxylic acid (or acids), followed by a quaternization with dimethyl sulfate: 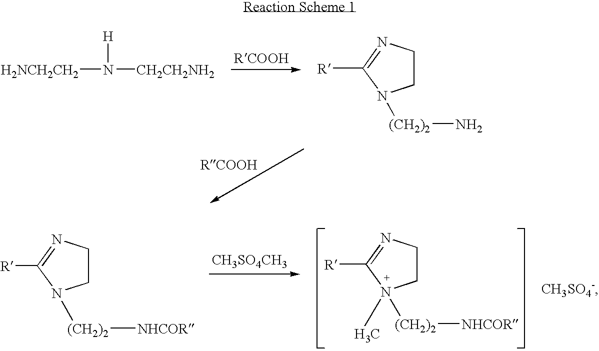
where R′ and R″ are alkyl groups having more than 10 carbon atoms, or groups containing alkyl radicals having more than 10 carbon atoms.

Likewise, Reaction Scheme 2 shows an example of the synthetic route for preparation of certain monoalkyl imidazoline quats, specifically, 1-methyl-1-(lower alkyl)-2-alkyl immidazolinium chlorides, via a reaction between N-lower alkyl-N-ethyleneamino diamine and a fatty carboxylic acid(s), followed by a quaternization with methyl chloride: 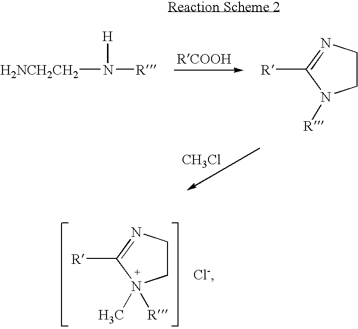
where R′ is an alkyl group or a group containing an alkyl radical having more than 10 carbon atoms, and R″′ is an alkyl group or a group containing an alkyl radical having 1 to 8 carbon atoms.

The Reaction Schemes 1 and 2 are non-limiting examples. Various other imidazoline quats may be obtained via different synthetic routes known to those skilled in the art and/or by varying the starting materials and the reactants in the examplyfied routes. For example, with respect to the synthetic route shown in the Reaction Scheme 1, the carboxylic acids R′COOH and R″COOH may be the same or different and/or may contain a variety of groups R′ and R″. The nature of R′ and R″ substitution in the carboxylic acids may be used to vary the R′ and R″ substituents in the resulting dialkyl imidazoline compounds. Likewise, the groups R′ and R″ may be varied in the route shown in the Reaction Scheme 2.

The carboxylic groups R′CO— and R″CO— may be derived from a variety of sources. Thus, essentially pure carboxylic acids may be used. The suitable carboxylic acids having C₁₈-C₂₄ alkyl groups include, for example, arachidic (C²⁰, including the carboxylic carbon¹, and 0 double bonds in the alkyl group (C²⁰:0) ), erucic (C²²:1), behemic (C²²:0) gadoleic (C²⁰:1), erucic (C²²:1), arachadonic (C²⁰:4), culpodonic (C²²:5), eicosapentaenoic (C²⁰:5), docosahexaenoic acid (C²²:6), tetrcosanoic (C²⁴:0); and nervonic (C²⁴:1). Other carboxylic acids, including acids having any desirable alkyl substitution may also be used.
¹ Subscripts with reference to the number of carbon atoms indicate the number of carbon atoms without the carboxylic group carbon; the superscripts indicate the number of carbon atoms of the carboxylic group including the carboxylic carbon. Thus, C²⁰ is C₁₉COO—.

The mixtures of quats may be obtained in any manner. For example, artificial mixtures of carboxylic acids may be used in reaction schemes 1 and 2. If a mixture of carboxylic acids is used, the reaction usually provides a corresponding mixture of compounds with R′/R″ substitution content similar or identical to the R′/R″ distribution in the mixture.

The use of artificial mixtures of pure carboxylic acids may not be economically feasible. Rather, the mixtures of carboxylic acids derived from a single source containing various carboxylic groups, such as natural or synthetic oils, triglycerides, and the like, are used. For example, such mixtures may be obtained in commercial quantities via saponification of ester-containing natural or synthetic substances.

In fact, carboxylic acids may be directly replaced in the reactions above by ester-containing natural or synthetic oil or a similar substance. Similarly to the use of carboxylic acid mixtures, the reaction between the ester-containing oil and a diamine or triamine usually provides a mixture of compounds with R′/R″ substitution content similar or identical to the R′/R″ distribution in the oil.

Preferably, the compositions of the invention contain compounds derived from natural and synthetic oils, fatty acids and/or triglycerides.

Thus, in yet another aspect, the invention provides a product of a reaction between

a) a compound of the formula 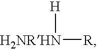
where R′ is C₁-C₃ alkylene, preferably —CH₂CH₂— group, and R is R′NH₂, C₁-C₃₀ alkyl, or C₁-C₃₀ alkylhydroxy, preferably. R is —CH₂CH₂NH₂ or —CH₂CH₂OH; and

b) a mixture of natural or synthetic oil-derived carboxylic acids or a natural or synthetic oil.

Oils that may be used directly or which may provide oil-derived mixtures of carboxylic acids include, for example, HEAR oil, cod liver oil, herring oil, menhaden oil, mustard seed oil, pilchard oil, hear oil, salmon oil, sardine oil and shark liver oil. Of course, other oils and similar substances may also be used. For listing of such substances, see 1 “Bailey's Industrial Oil and Fat Products” (Daniel Swern, John Wiley & Sons, 4th Ed. 1979), at pages 416-417, 447, 449-450, and 452, which are hereby incorporated by reference.

The substitution content of any quat mixture, including those derived from the natural or synthetic oils, may be relatively easily characterized in terms of the weight percentages of certain substituents in the mixture. Any analytical methods known to those skilled in the art may be used, such as High Performance Liquid Chromatography or Gas Chromatography, where suitable, to determine the identity and the weight proportions of the quat components in the mixture. On the basis of such analysis, the “substitution content” of the mixture as defined herein (e.g., C₁₆₋₂₄ or C₂₄₋₂₄ contents), may be easily derived from the weight percentages and molecular weights of the quat components by using the calculation methodology described herein.

Table 11 shows known approximate weight percentages of some of the C²⁰+ components in some of the common oils:

TABLE 11

| Substance | C²⁰:0 | C²⁰:1 | C²⁰:4 | C²⁰:5 | C²²:0 | C²²:1 | C²²:5 | C²²:6 | C²⁴:0 |
|---|---|---|---|---|---|---|---|---|---|
| Cod liver | 8.8-14.6% | | | 2.6-9% | | 4.6-13.3% | 1-2% | 8.6-19% | |
| oil | | | | | | | | | |
| Herring | | 1.5-19.2% | | 4.6-10.2% | | 2.8-19.9% | 1-3.7% | 3.8-24.1% | |
| oil | | | | | | | | | |
| Menhaden | | 0.9-2.7% | 0.6-1.2% | 10.2-13.5% | | 0.7-1.7% | 1.1-2.3% | 3.3-14% | |
| oil | | | | | | | | | |
| Pilchard | | 3.2% | 1.6% | 16.9% | | 3.6% | 2.5% | 12.9% | |
| (Sardine) | | | | | | | | | |
| oil | | | | | | | | | |
| HEAR oil | | 0.8-13.5% | | | | 20.1-59.4% | | | 0.1-1.4% |
| Mustard | | 7% | | | | 44.2% | | | |
| Seed oil | | | | | | | | | |

The oils shown in Table 11 generally contain from about 30% to about 90% of C₂₀-C₃₀ alkyl groups in their fatty carboxylic groups by weight. The oils often exhibit substantial variations in C₂₀-C₃₀ content, and also include some C₁-C₁₉ content.

Thus, the more preferred mixtures of dialkyl quats are derived from rapeseed oil, especially high erucic rapeseed oil (HEAR oil), which typically contains 46% of C²²:1 alkyl (erucic), 1.5% of C²²:0 alkyl (behemic), and 11% of C²⁰:1 alkyl (gadoleic) by weight. Yet more preferred are qtars derived from hydrogenated HEAR oil in which the double bonds of the erucic is hydrogenated, resulting in behenic (C²²:0). HEAR oil or HEAR oil-derived mixtures of carboxylic acids may be used to obtain quat mixtures. The dialkyl imidazoline quat mixture obtained in this manner is preferred. It is also referred to in the examples herein as di-behenyl immidazolinium methosulfate, reflecting the prevalence of C²²:1 alkyls.

The compositions containing imidazoline quats may in the form of quat raw materials.

In general, a producer provides raw quats to manufacturers of personal care and cosmetic products, who formulate them in the final products. An important characteristic of raw quats, as well as the final products that incorporate them, is the so-called cationic activity, which measures a concentration of positive charges in a substance, product, etc. The cationic activity may be measured by several methods readily understood by those skilled in the art. One such method utilizes a standardized solution of an anionic material, such as sodium lauryl sulfate. This material is added to the solution containing the quat until full complexation of the quat's cations (the end point) has been reached. The end point can be measured potentiometrically or by the use of color indicators.

Typical tests involve titrating a sample of the quat, usually dissolved in a solvent, with the standardized solution of sodium lauryl sulfate until the endpoint is reached. As described in the co-pending and co-assigned U.S. patent application Ser. No. 09/438,631, incorporated by reference herein in its entirety, once the endpoint is reached, the cationic activity can be calculated according to the following formula:

$$\% cationic activity = \frac{mL \times N \times MW \times 100}{S . wt . \times 1000}$$

Where:
mL=the number of mL of anionic material
N=the normality of the solution used
MW=the molecular weight of the quat being analyzed
S.wt.=the sample weight in grams

For additional information regarding the methodology for measuring the cationic activity, see W. Schempp and H. T. Trau, Wochenblatt fur. Papierfabrikation 19, 1981, pages 726-732, or J. P. Fischer and K. Lohr, Organic Coatings Science Technology, Volume 8, pages 227-249, Marcel Dekker, Inc. April 1986), both incorporated herein by reference in their entirety.

It is desirable to provide raw quats in a concentrated form with high cationic activity, as a solid or semi-solid solution or dispersion. Without wishing to be bound by any specific theory, it is believed that a desired amount of a given quat or mixture of quats to be placed in a formulation may be measured by the cationic activity of the quat raw material. The quat raw materials with high cationic activity permit better transportation efficiency since they occupy smaller space while providing the same desired quat amounts. It is also desirable to produce raw quats that, in addition to having high cationic activity, provide for ease in commercial handling and storage. For example, the raw quat that melt at lower temperatures minimize quat decomposition and improve energy efficiency. For this purpose, it is preferred for the raw quats to be flakeable or pastillatable.

Thus, the composition containing imidazoline quats or quat mixtures described herein may be in the form of concentrated, usually solid, solutions or suspensions of the quat(s) or mixture(s) in a suitable carrier. Such compositions are called herein quats raw materials. The preferred carrier is a solvent, and the preferred solvents include isopropyl alcohol, SDA-40, propylene glycol, butylenes glycol, various fatty alcohols, and mixtures thereof. Preferably, the quat raw materials of the invention are flakeable or pastillatable solids with high quat cationic activity. The quat cationic activity is the cationic activity that is attributed to quaternary nitrogen compounds. The preferred total quat cationic activity of the quat raw materials of the invention is greater than 10%, preferably, greater than 20%, more preferably, greater than 35%, yet more preferably, greater than 50%.

The quat raw materials of the invention may include one or more desirable ingredients of final cosmetic/personal care formulations, such as emollients and the like, as well as various impurities. The list of possible ingredients may be found below.

The compositions containing imidazoline quats and/or quat mixtures may also be in the form of various cosmetic and/or personal care products. Such compositions may be referred to as final product compositions. Examples of the final product compositions include sunscreen compositions for hair and/or skin, such as lotions, gels, sprays, and the like, hand cleaners, bath compositions, suntan oils, anti-perspirant compositions, perfumes and colognes, cold creams, pre-shaves, deodorants, topical pharmaceutical ointments, skin moisturizers, facial cleansers, cleansing creams, skin gels, shampoos, hair conditioners, detergents, household cleaning products, make-up products, lipstick products, mascara, and hair coloring products. The preferred final product compositions of the invention are compositions for treating human hair, such as shampoos or conditioners.

The final product compositions including preparations for skin and hair, include imidazoline quats or imidazoline quat mixtures described herein. The amount of imidazoline quats or mixtures in the products depend on the specific application, and may vary from about 0.1% to about 40%, more preferably, from about 0.1% to about 10%, yet more preferably, from about 0.5% to about 2% by the weight of the product composition. However, different amounts of imidazoline quats or imidazoline quat mixtures may be preferred depending on the nature of the product.

The final product compositions that include the imidazoline quats or mixtures of imidazoline quats may be in the form of liquids, gels, creams, emulsions, foams, and solids; may be clear or opaque; and may be formulated as aqueous and non-aqueous preparations, including but not limited to topical preparations. Preferably, the final product compositions are dispersions or solutions in water, or in a mixture of water with a suitable secondary solvent. Suitable inert solvents include various lower alkanols and glycols. Lower alkanols having from one to four carbon atoms are suitable for use with the present invention, and lower alkanols having from two to three carbon atoms are preferred. Glycols having from three to eight carbon atoms are suitable for use with the present invention, while glycols having from three to six carbon atoms are preferred. Examples of suitable lower alkanols and glycols include methanol, ethanol, isopropanol, butanol, hexylene glycol, 1,3-butylene glycol, 1,2- and 1,3-propane diol, 2-methyl 1,3-propane diol, propylene glycol, diethylene glycol, and the like. The total amount of solvent may be up to about 98% by weight of the composition, preferably, from about 20% to about 90%, more preferably, from about 50% to about 90% by weight of the composition. Again, however, different amounts of solvent may be preferred depending on the nature of the product. If a mixture of water and a secondary solvent is used, the secondary solvent may be present in the amount of up to 90%, preferably, between about 25% and about 80% by weight of water in the composition.

In addition to the imidazoline quaternary compounds, the final product compositions may include various active and additional ingredients, both conventional and otherwise. Of course, a decision to include an ingredient and the choice of specific active and additional ingredients depends on the specific application and product formulation. Also, the line of demarcation between an “active” ingredient and an “additional ingredient” is artificial and dependent on the specific application and product type. A substance that is an “active” ingredient in one application or product may be an “additional” ingredient in another, and vice versa.

The final product compositions may include one or more active ingredients, which provide some benefit to the object of the application of the composition, for example, hair or skin. Such active ingredients may include one or more substances such as cleaning agents, hair conditioning agents, skin conditioning agents, hair styling agents, antidandruff agents, hair growth promoters, perfumes, sunscreen compounds, pigments, moisturizers, film formers, hair colors, make-up agents, detergents, thickening agents, emulsifiers, antiseptic agents, deodorant actives, surfactants and pharmaceuticals useful for topical purposes for transdermal delivery.

The choice of the active ingredient(s) depends on the nature of the desired cosmetic or personal care product. For example, the sunscreen compounds may be used in the sunscreen lotions, shampoos, medicated shampoos, hair care lotions and the like. For each type of active ingredient, one or more compounds may be present. Likewise, more than one type of active ingredient may be present.

It is believed that imidazoline quat and/or quat mixtures improves hair substantivity of hydrophobic ingredients of cosmetic and personal care products, which is typically thought of as the degree of deposition of the hydrophobic ingredient on hair and is desirable. The hydrophobic components are those that are substantially insoluble in water. Typically, such hydrophobic ingredients are soluble in oils. Thus, the compositions described herein may further include at least one hydrophobic ingredient, examples of which include botanical extracts, vitamin E, vitamin A, silicones, waxes and antioxidants.

### Surfactants

In addition to the imidazoline quaternary compounds and/or mixtures, other surfactants may be present in the compositions described herein, including one or more nonionic surfactants, anionic surfactants, cationic surfactants, amphoteric surfactants, zwitterionic surfactants, and mixtures thereof. For some of surfactants that may be used in combination with the compositions of the invention, please see McCutcheon's, Detergents and Emulsifiers, (1986), U.S. Pat. Nos. 5,151,210, 5,151,209, 5,120,532, 5,011,681, 4,788,006, 4,741,855, U.S. Pat. Nos.. 4,704,272, 4,557,853, 4,421,769, 3,755,560; all incorporated herein by reference in their entirety.

Cationic Surfactants

Many cationic surfactants are known in the art, and almost any cationic surfactant having at least one long chain alkyl group of about 10 to 24 carbon atoms is suitable in the present invention.

Ammonium Quats

The compositions of the invention may include quaternary ammonium cationic surfactants of the formula 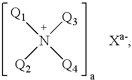
where X and a are as previously described, Q₁ is C₁₂-C₂₂ alkyl, C₁₂-C₂₂ alkyl amido C₁-C₆ alkylene, C₁₂-C₂₂ alkylhydroxy; Q₂ is C₁₂-C₂₂ alkyl, C₁₂-C₂₂ alkyl amido. C₁-C₆ alkylene, C₁₂-C₂₂ alkylhydroxy, benzyl, or C₁-C₆ alkyl; Q₃ and Q₄ are independently C₁-C₆ alkyl or benzyl.

Examples of suitable quaternary ammonium surfactants include cetyl ammonium chloride cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and mixtures thereof.

Additional quaternary ammonium salts include those wherein the C₁₂-C₂₂ alkyl is derived from a tallow fatty acid or from a coconut fatty acid. Examples of quaternary ammonium salts derived from these tallow and cococut sources include ditallow dimethyl ammonium chlroide, ditallow dimehtyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

More preferred quaternary ammonium surfactants are dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Fatty Amines

The compositions of the invention may also include salts of primary, secondary and tertiary C₁₂-C₂₂ amines. Examples of such suitable amines include stearamido propyl dimethyl amine, diethyl amino ethyl stearamide, dimethyl stearamine, dimethyl soyamine, soyamine, tri(decyl)amine, ethyl stearylamine, ethoxylated stearylamine, dihydroxyethyl stearylamine, and arachidylbehenylamine. Suitable amine salts include the halogen, acetate, phosphate, nitrate, citrate, lactate and alkyl sulfate salts. Such salts include stearylamine hydrochloride, soyamine chloride, stearylamine formate, N-tallowpropane diamine dichloride and stearamidopropyl dimethylamine citrate. Some cationic amine surfactants useful in the compositions of the present invention are disclosed in U.S. Pat. No. 4,275,055, incorporated by reference herein.

Non-Ionic Surfactants

The compositions of the invention may also include various non-ionic surfactants. Among the suitable nonionic surfactants are condensation products of C₈-C₃₀ alcohols with sugar or starch polymers. These compounds can be represented by the formula (S)ₙ—O—R, wherein S is a sugar moiety such as glucose, fructose, mannose, and galactose; n is an integer of from about 1 to about 1000, and R is C₈-C₃₀ alkyl. Examples of suitable C₈-C₃₀ alcohols from which the R group may be derived include decyl alcohol, cetyl alcohol, stearyl alcohol, lauryl alcohol, myristyl alcohol, oleyl alcohol, and the like. Specific examples of these surfactants include decyl polyglucoside and lauryl polyglucoside.

Other suitable nonionic surfactants include the condensation products of alkylene oxides with fatty acids (i.e., alkylene oxide esters of fatty acids). These materials have the general formula RCO(X)ₙ OH, wherein R is a C₁₀-C₃₀ alkyl, X is —OCH₂CH₂— (derived from ethylene oxide) or —OCH₂CHCH₃— (derived from propylene, oxide), and n is an integer from about 1 to about 200.

Yet other suitable nonionic surfactants are the condensation products of alkylene oxides with fatty acids (i.e., alkylene oxide diesters of fatty acids) having the formula RCO(X)ₙOOCR, wherein R is a C₁₀-C₃₀ alkyl, X is —OCH₂CH₂— (derived from ethylene oxide) or —OCH₂CHCH₃— (derived from propylene oxide), and n is an integer from about 1 to about 200.

Yet other nonionic surfactants are the condensation products of alkylene oxides with fatty alcohols (i.e., alkylene oxide ethers of fatty alcohols) having the general formula R(X)ₙOR′, wherein R is C₁₀-C₃₀ alkyl, n is an integer from about 1 to about 200, and R′ is H or a C₁₀-C₃₀ alkyl.

Still other nonionic surfactants are the compounds having the formula RCO(X)ₙOR′ wherein R and R′ are C₁₀-C₃₀ alkyl, X is —OCH₂CH₂— (derived from ethylene oxide) or —OCH₂CHCH₃— (derived from propylene oxide), and n is an integer from about 1 to about 200.

Examples of alkylene oxide-derived nonionic surfactants include ceteth-1, ceteth-2, ceteth-6, ceteth-10, ceteth-12, ceteraeth-2, ceteareth6, ceteareth-10, ceteareth-12, steareth-1, steareth-2, stearteth-6, steareth-10, steareth-12, PEG-2 stearate, PEG4 stearate, PEG6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful nonionic surfactants include polyhydroxy fatty acid amides disclosed, for example, in U.S. Pat. Nos. 2,965,576, 2,703,798, and 1,985,424, which are incorporated herein by reference.

Anionic Surfactants

The compositions of the invention may also include various anionic surfactants. Several examples of suitable anionic surfactants are disclosed in U.S. Pat. No. 3,929,678, which is incorporated herein by reference. Further examples of suitable anionic surfactants include alkoyl isethionates, and alkyl ether sulfates.

The alkoyl isethionates typically have the formula RCO—OCH₂CH₂— SO₃M, wherein R is C₁₀-C₃₀ alkyl, and M is a water-soluble cation, such as ammonium, sodium, potassium, or triethanolamine. The examples of suitable isethionates include ammonium cocoyl isethionate, sodium cocoyl isethionate, sodium lauroyl isethionate, sodium stearoyl isethionate, and mixtures thereof. Preferred for used herein are ammonium cocoyl isethionate, sodium cocoyl isethionate, and mixtures thereof.

The alkyl ether sulfates typically have the formulas ROSO₃M and RO(C₂ H₄O)ₓSO₃M, where R is C₁₀-C₃₀ alkyl, x varies from about 1 to about 10, and M is a water-soluble cation such as ammonium, sodium, potassium and triethanolamine.

Yet another suitable class of anionic surfactants are alkali metal salts of C₈-C₃₀ carboxylic acids and alkylsulfonates of the formula R₁—SO₃M (where R₁ is C₈-C₃₀ alkyl; preferably, C₁₂-C₂₂ alkyl, and M is a cation), including succinamates, and C₁₂-C₂₄ olefin sulfonates and carboxylates.

Amphoteric Surfactants

The compositions of the invention may also include zwitterionic and amphoteric surfactants. Suitable amphoteric and zwitterionic surfactants are, for example, derivatives of mono- or di-C₈-C₂₄ secondary and tertiary amines, such as alkyl imino acetates, carboxylates, sulfonates, sulfates, phosphates, and phosphonates, including iminodialkanoates and aminoalkanoates of the formulas RN(CH₂)ₘ CO₂ M₂ and RNH(CH₂)ₘ CO₂M, where m varies from 1 to 4, R is C₈-C₃₀ alkyl; preferably, C₁₂-C₂₂ alkyl, and M is H, alkali metal, alkaline earth metal ammonium, or alkanolammonium.

Other suitable amphoteric and zwitterionic surfactants are imidazolinium and ammonium derivates. Suitable examples of such amphoteric surfactants include sodium 3-dodecyl-aminopropionate, sodium 3-dodecylaminopropane sulfonate, N-alkyltaurines; N-higher alkyl aspartic acids, and coamidopropyl PG-dimonium chloride phosphate. For further examples of suitable amphoteric and zwitterionic surfactants, please see U.S. Pat. Nos. 2,658,072, 2,438,091, and 2,528,378, which are incorporated herein by reference

Yet other suitable amphoteric and zwitterionic surfactants are betaines. Examples of suitable betaines include coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and amidobetaines and amidosulfobetaines, oleyl betaine, and cocamidopropyl betaine.

Sunscreen Compounds

A wide variety of sunscreen compounds are suitable for use with the compositions of the present invention. Depending on the nature of the composition, the sunscreen compounds may be added in the amount of up to about 40% by weight of the composition, preferably, from about 1% to about 30%. However, the preferred amount may vary depending on the nature of the composition. Thus, for the final product compositions in the form of a shampoo or conditioner, the suitable sunscreen agent may be included in the amount of up to about 40% by weight of the composition, preferably, from about 0.5% to about 5%, more preferably, from about 05 to about 1.5% by weight of the composition. Suitable sunscreen compounds include, for example, p-aminobenzoic acid, its salts and its derivatives; anthranilates; salicylates; cinnamic acid derivatives; dihydroxycinnamic acid derivatives; trihydroxycinnamic acid derivatives; hydrocarbons; dibenzalacetone and benzalacetophenone; naphtholsulfonates; dihydroxy-naphtholic acid and its salts; coumarin derivatives; diazoles; quinine salts; quinoline derivatives; hydroxy- or methoxy-substituted benzophenones; uric and vilouric acids; tannic acid and its derivatives; hydroquinone; amino benzoates, salicylates, ferrulic acid derivatives, phenylbenzimidazole sulfonic acids, benzophenone sulfonic acids, thioctic acids derivatives, oil-soluble cinnamates, and benzophenones. For other suitable sunscreen compounds, please see Segarin, et al., Cosmetics Science and Technology, Chapter VIII, pages 189 et seq., incorporated herein by reference.

Specific suitable sunscreen compounds include 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl-4->bis(hydroxypropyl)!-aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl p-dimethylaminobenzoate, 2-phenylbenzimidazole-5-sulfonic acid, 2-(p-dimethyl-aminophenyl)-5-sulfonicbenzoxazoic acid, para-aminobenzoic acid, benzophenone-1, benzophenone-1, benzophenone-2, benzophenone-3, benzophenone-4, benzophenone-5, benzophenone-6, benzophenone-8, benzophenone-12, methoxycinnamate, avobenzone, ethyl dihydroxypropyl para-aminobenzoate, glyceryl para-aminobenzoate, methyl anthranilate, octocrylene, octyl dimethyl para-aminobenzoate, octyl methoxycinnamate, octyl salicylate, zinc oxide, titanium dioxide, and red petrolatum.

Emollients

The compositions of the invention may also include one or emollient compounds such as fats, waxes, lipids, silicones, hydrocarbons, fatty alcohols and a wide variety of solvent materials. The amount of the emollient depends on the application. For the final product compositions, emmollinets are included in the amount of up to 50% by weight of the composition, preferably, from about 0.1% to about 20%, and more preferably, from about 0.5% to about 10% by weight of the composition.

Examples of suitable emollients include C₈-₃₀ alkyl esters of C₈-₃₀ carboxylic acids; C₁₋₆ diol monoesters and diesters of C₈₋₃₀ carboxylic acids; monoglycerides, diglycerides, and triglycerides of C₈₋₃₀ carboxylic acids, cholesterol esters of C₈₋₃₀ carboxylic acids, cholesterol, and hydrocarbons. Examples of these materials include diisopropyl adipate, isopropyl myristate, isopropyl palmitate, ethylhexyl palmitate, isodecyl neopentanoate, C₁₂₋₁₅ alcohols benzoates, diethylhexyl maleate, PPG-14 butyl ether, PPG-2 myristyl ether propionate, cetyl ricinoleate, cholesterol stearate, cholesterol isosterate, cholesterol.acetate, jojoba oil, cocoa butter, shea butter, lanolin, lanolin esters, mineral oil, petrolatum, and straight and branched C₁₆-C₃₀ hydrocarbons.

Also useful are straight and branched chain fatty C₈-C₃₀ alcohols, for example, stearyl alcohol, isostearyl alcohol, ehenyl alcohol, cetyl alcohol, isocetyl alcohol, and mixtures thereof. Examples of other suitable emollients are disclosed in. U.S. Pat. No. 4,919,934; which is incorporated herein by reference in its entirety.

Other suitable emollients are various alkoxylated ethers, diethers, esters, diesters, and trimesters. Examples of suitable alkoxylated ethers include PPG-10 butyl ether, PPG-11 butyl ether, PPG-12 butyl ether, PPG-13 butyl ether, PPG-14 butyl ether, PPG-15 butyl ether, PPG-16 butyl ether, PPG-17 butyl ether, PPG-18 butyl ether, PPG-19 butyl ether, PPG-20 butyl ether, PPG-22 butyl ether, PPG-24 butyl ether, PPG-30 butyl ether, PPG-11 stearyl ether, PPG-15 stearyl ether, PPG-10 oleyl ether, PPG-7 lauryl ether, PPG-30 isocetyl ether, PPG-10 glyceryl ether, PPG-15 glyceryl ether, PPG-10 butyleneglycol ether, PPG-15 bttylene glycol ether, PPG-27 glyceryl ether, PPG-30 cetyl ether, PPG-28 cetyl ether, PPG-10 cetyl ether, PPG-10 hexylene glycol ether, PPG-15 hexylene glycol ether, PPG-10 1,2,6-hexanetriol ether, PPG-15 1,2,6-hexanetriol ether, and mixtures thereof.

Examples of alkoxylated diethers include PPG-10 1,4-butanediol diether, PPG-12 1,4-butanediol diether, PPG-14 1,4-butanediol diether, PPG-2 butanediol diether, PPG-10 1,6-hexanediol diether, PPG-12 1,6-hexanediol diether, PPG-14 hexanediol diether, PPG-20 hexanediol diether, and mixtures thereof. Preferred are those selected from the group consisting of PPG-10 1,4-butanediol diether, PPG-12 1,4-butanediol diether, PPG-10 1,6-hexandiol diether, and PPG-12 hexanediol diether, and mixtures thereof.

Examples of suitable alkoxylated diesters and trimesters are disclosed in U.S. Pat. Nos. 5,382,377, 5,455,025 and 5,597,555, assigned to Croda Inc., and incorporated herein by reference.

Suitable lipids include C₈-C₂₀ alcohol monosorbitan esters, C₈-C₂₀ alcohol sorbitan diesters, C₈-C₂₀ alcohol sorbitan triesters, C₈-C₂₀ alcohol sucrose monoesters, C₈-C₂₀ alcohol sucrose diesters, C₈-C₂₀ alcohol sucrose triesters, and C₈-C₂₀ fatty alcohol esters of C₂-C₆₂-hydroxy acids. Examples of specific suitable lipids are sorbitan diisostearate, sorbitan dioleate, sorbitan distearate, sorbitan isosotearate, sorbitan laurate, sorbitan oleate, sorbitan palmitate, sorbitan sesquioleate, sorbitan esquistearte, sorbitan stearate, sorbitan triiostearte, sorbitan trioleate, orbitan tristeate, sucrose cocoate, sucrodilaurate, sucrose distearate, sucrose laurate, sucrose myristate, sucrose oleate, sucrose palmitate, sucrose ricinoleate, sucrose stearate, sucrose tribehenate, sucrose tristearate, myristyl lactate, stearyl lactate, isostearyl lactate, cetyl lactate, palmityl lactate, cocoyl lactate, and mixtures thereof.

Other suitable emollients include mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearte, sorbitan dilaurate, sorbitan stearate, sorbitan laurate, sucrose laurate, sucrose dilaurate, sodium isostearyl lactylate, lauryl pidolate, sorbitan stearate, stearyl acohol, cetyl alcohol, behenyl alcohol, PPG-14 butyl ether, PPG-15 stearyl ether, and mixtures thereof.

### Emulsifiers

The compositions of the invention may also include various emulsifiers. In the final product compositions of the invention, emulsifiers may be included in the amount of up to about 10%, preferably, in the amount of from about 0.5% to about 5% by weight of the composition. The examples of suitable emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, polyethyleneglycols, polypropyleneglyocis, and mixtures thereof.

Anti-Dandruff

The compositions of the invention may also include antidandruff agents. The examples of suitable antidandruff agents include zinc pyrithione, sulphur, and selenium sulfide.

Hair Oxidizers

The compositions of the invention may also include hair oxidizing/reducing agents. The examples of suitable hair oxidizing/reducing agents include hydrogen peroxide, perborate, thioglycolates and persulfate salts.

Thickeners

The compositions of the invention may also include various thickeners, such as cross-linked acrylates, nonionic polyacrylamides, xanthan gum, guar gum, gellan gum, and the like; polyalkyl siloxanes, polyaryl siloxanes, and aminosilicones. In the final product compositions of the invention, thickeners may be included in the amount of up to about 10%, preferably, in the amount of from about 0.2% to about 5% by weight of the composition.

The specific examples of the suitable thickening silicon compounds include polydimethylsiloxane, phenylsilicone, polydiethylsiloxane, and polymethylphenylsiloxane. Some of the suitable silicon compounds are described in European Patent Application EP 95,238 and U.S. Pat. No. 4,185,017, which are incorporated herein by reference. The compositions of the invention may also include silicone polymer materials, which provide both style retention and conditioning benefits to the hair. Such materials are described in U.S. Pat. No. 4,902,499, which is incorporated herein by reference.

Hair Conditioning Agents

The compositions of the invention may also include hydrolyzed animal protein hair conditioning agents. Croda Incorporated sells an example of a commercially available material under the tradename Crotein Q-RTM. Other examples include urea, glycerol, and propoxylated glycerols, including those described in U.S. Pat. No. 4,976,953, which is incorporated by reference herein.

Hair Setting Agents

The compositions of the invention may also include a hair setting agent to impart styling benefits upon application to hair. The hair setting polymers may be homopolymers, copolymers, terpolymers, etc. For convenience in describing the polymers hereof, monomeric units present in the polymers may be referred to as the monomers from which they can be derived. The monomers can be ionic (e.g., anionic, cationic, amphoteric, zwitterionic) or nonionic.

Examples of anionic monomers include unsaturated carboxylic acid monomers such as acrylic acid, methacrylic acid, maleic acid, maleic acid half ester, itaconic acid, fumaric acid, and crotonic acid; half esters of an unsaturated polybasic acid anhydride such as succinic anhydride, phthalic anhydride or the like with a hydroxyl group-containing acrylate and/or methacrylate such as hydroxyethyl acrylate and, hydroxyethyl methacrylate, hydroxypropyl acrylate and the like; monomers having a sulfonic acid group such as styrenesulfonic acid, sulfoethyl acrylate and methacrylate, and the like; and monomers having a phosphoric acid group such as acid phosphooxyethyl acrylate and methacrylate, 3-chloro-2-acid phosphooxypropyl acrylate and methacrylate, and the. like.

Examples of cationic monomers include monomers derived from acrylic acid or methacrylic acid, and a quaternarized epihalohydrin product of a trialkylamine having 1 to 5 carbon atoms in the alkyl such as (meth)acryloxypropyltrimethylammonium chloride and (meth)acryloxypropyl-triethylammonium bromide; amine derivatives of methacrylic acid or amine derivatives of methacrylamide derived from methacrylic acid or methacrylamide and a dialkylalkanolamine having C₁-C₆ alkyl groups such as dimethylaminoethyl (meth)acrylate, diethylaminoethyl (meth)acrylate, dimethylaminopropyl (meth)acrylate, or dimethylaminopropyl (meth)acrylamide.

Examples of the amphoteric monomers include zwitterionized derivatives of the aforementioned amine derivatives of (meth)acrylic acids or the amine derivatives of (meth)acrylamide such as dimethylaminoethyl (meth)acrylate, dimethylaminopropyl(meth)acrylamide by a halogenated fatty acid salt such as potassium monochloroacetate, sodium monobromopropionate, aminomethylpropanol salt of monochloroacetic acid, triethanolamine salts of monochloroacetic acid and the like; and amine derivatives of (meth)acrylic acid or (meth)acrylamide, as discussed above, modified with propanesultone.

Examples of nonionic monomers are acrylic or methacrylic acid esters of C₁-C₂₄ alcohols, such as methanol, ethanol, 1-propanol, 2-propanol, 1-butanol, 2-methyl-1-propanol, 1-pentanol, 2-pentanol, 3-pentanol, 2-methyl-1-butanol, 1-methyl-1-butanol, 3-methyl-1-butanol, 1-methyl-1-pentanol, 2-methyl-1-pentanol, 3-methyl-1-pentanol, t-butanol, cyclohexanol, 2-ethyl-1-butanol, 3-heptanol, benzyl alcohol, 2-octanol, 6-methyl-1-heptanol, 2-ethyl-1-hexanol, 3,5-dimethyl-1-hexanol, 3,5,5-trimethyl-1-hexanol, 1-decanol, 1-dodecanol, 1-hexadecanol, 1-octadecanol, styrene; chlorostyrene; vinyl esters such as vinyl acetate; vinyl chloride; vinylidene chloride; acrylonitrile; alpha-methylstyrene; t-butylstyrene; butadiene; cyclohexadiene; ethylene; propylene; vinyl toluene; alkoxyalkyl (meth)acrylate, methoxy ethyl (meth)acrylate, butoxyethyl (meth)acrylate; allyl acrylate, allyl methacrylate, cyclohexyl acrylate and methacrylate, oleyl acrylate and methacrylate, benzyl acrylate and methacrylate, tetrahydrofurfuryl acrylate and methacrylate, ethylene glycol di-acrylate and -methacrylate, 1,3-butyleneglycol di-acrylate and -methacrylate, diacetonacrylamide, isobornyl (meth)acrylate, n-butyl methacrylate, isobutyl methacrylate, 2-ethylhexyl methacrylate, methyl methacrylate, t-butylacrylate, t-butylmethacrylate, and mixtures thereof.

Examples of anionic hair styling polymers are copolymers of vinyl acetate and crotonic acid, terpolymers of vinyl acetate, crotonic acid and a vinyl ester of an alpha-branched saturated aliphatic monocarboxylic acid such as vinyl neodecanoate; and copolymers of methyl vinyl ether and maleic anhydride, acrylic copolymers and terpolymers containing acrylic acid or methacrylic acid.

Examples of cationic hair styling polymers are copolymers of amino-functional acrylate monomers such as lower alkylamino alkyl acrylate or methacrylate monomers such as dimethyl aminoethylmethacrylate with compatible monomers such as N-vinylpyrrolidone or alkyl methacrylates such as methyl methacrylate and ethyl methacrylate and alkyl acrylates such as methyl acrylate and butyl acrylate.

Miscellaneous Components

The compositions of the invention may also include a wide range of miscellaneous ingredients. Some suitable miscellaneous ingredients commonly used in the cosmetic and personal care industry are described in The CTFA Cosmetic Ingredient Handbook, (2^{nd} Ed., 1992), which is incorporated by reference herein.

Thus, the compositions of the invention may also include one or more absorbents, anti-acne agents, anti-perspirants, anticaking agents, antifoaming agents, antimicrobial agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, humectants, keratolytics, opacifying agents, pH adjusters, preservatives, propellants, proteins, retinoids, reducing agents, sequestrants, skin bleaching agents, skin-conditioning agents (humectants, miscellaneous, and occulsive), skin soothing agents, skin healing agents, softeners, solubilizing agents, lubricants, penetrants, plastisizers, solvents and co-solvents, sunscreening additives, salts, essential oils, and vitamins.

The examples of suitable pH adjusters include sodium hydroxide, triethanoleamine, and aminomethylpropanol, and mixtures thereof. If pH adjusters are present in a final product composition, the amount may vary from about 0.01% to about 5%, preferably, from about 0.1% to about 2% by weight of the composition.

The examples of suitable film formers include glycerin/diethylene glycol myrystate copolymer, glycerin/diethylene glycol adipate copolymer, ethyl ester of PVM/MA copolymer, PVP/dimethiconylacrylate/polycarbamyl/polyglycol ester, and mixtures thereof. If the film formers are present in the final product compositions, the amount may vary from about 0.1% to about 15.0% by weight of the composition, preferably, from about 0.1% to about 2.5% by weight of the composition.

The examples of suitable vitamins include tocopherol, tocopherol acetate, retinoic acid, retinol, and retinoids.

The examples of suitable anti-acne medicaments include resorcinol, sulfur, salicylic acid, erythromycin, zinc, and benzoyl peroxide.

The examples of suitable skin bleaching or lightening agents include hydroquinone, and kojic acid. The examples of suitable aesthetic components such as fragrances, pigments, colorings, and the like, include panthenol and derivatives (e.g., ethyl panthenol), aloe vera, pantothenic acid and its derivatives, clove oil, menthol, camphor, eucalyptus oil, eugenol, menthyl lactate, witch hazel distillate, allantoin, bisabolol, and dipotassium glycyrrhizinate.

A particularly preferred aspect of the present invention is a mixture of dialkyl imidazoline quats having a structure illustrated in formula II. This does not exclude the presence of other quats including monoalkyl imidazoline quats, dialkyl quats and the like. However, these mixtures include at least two distinct dialkyl imidazoline quats, each having a general structure illustrated in formula II. In this particularly preferred aspect of the invention, m is 1 or 2, n is 1-3 and preferably 2 or 3. and R⁹ is C₁-C₆ alkyl group or a benzyl group, either of which may be substituted or unsubstituted. R¹⁰ is preferably hydrogen or a C₁-C₆ alkyl group which may additionally be hydroxy substituted and/or saturated or unsaturated. X and a in all of this aspect are previously described in connection with formula II.

R⁸ and R¹¹ may be the same or different and at least 50% of the R⁸ and R¹¹ groups of the mixture of dialkyl imidazoline quats having a general structure illustrated in formula II, are C₁₇-C₃₅ alkyl groups. These may be saturated or unsaturated and/or hydroxy substituted or unsubstituted. These groups may also be strait, branched or cyclized. It should be noted when molecules such as those depicted in formula II are manufactured, in accordance with the present invention, an alkyl chain having 18 carbons in length may be used as, for example R⁸. However, a terminal carbon may, depending upon the process used, actually become a carbon within the diazole ring. The resulting chain extends from the ring for only 17 carbons. Similarly, if a C₃₆ carbon chain were used to form the dialkyl imidazoline quats of formula II, R⁸ might appear, as illustrated, to have only 35 carbons.

It is more preferable that at least about 70% of the R⁸ and R¹¹ groups in the mixture of dialkyl imidazoline quats of formula II are C₁₇-C₃₅ alkyl groups, substituted or unsubstituted and/or unsaturated or unsatureated as previously described. These groups may also be straight, branched or cyclized.

In another preferred embodiment of this aspect of the present invention, the mixture of dialkyl imidazoline quats includes two or more dialkyl imidazoline quats having the basic structure found in formula II. In this embodiment, at least about 30% of these dialkyl imidazoline quats are di-substituted with alkyl groups having chain lengths of 19 carbons or longer. That is, for at least 30% of the dialkyl imidazoline quats of the mixture having the general structure of formula II, both R⁸ and R¹¹ are alkyl groups of at least 19 carbons in length. Preferably, these groups are are C₁₉-C₃₅ alkyl groups, substituted or unsubstituted and/or unsaturated or unsatureated as previously described. These groups may also be straight, branched or cyclized. Particularly preferred are mixtures in which the alkyl groups discussed immediately above are C₁₉-C₂₉ alkyl groups and even more preferably C₁₉-C₂₆ alkyl groups More preferably, at least 40% of the dialkyl imidazoline quats having the general formula II in the mixture are di-substituted with C₁₉ groups or larger and most preferably at least 50% are di-substituted in this fashion.

One material which can be used in the production of mixtures of dialkyl imidazoline quats having the general formula II is hydrogenated or non-hydrogenated HEAR oil. HEAR oil typically contains about 3% C₁₆, 39% C₁₈, 9% C₂₀, 47% C₂₂ and 2% C₂₄ fatty acid substituents. When this material is used as the source of the alkyl groups in the dialkyl imidazoline quats having the general formula II of this aspect of the invention, the result is a significant percentage of all of the dialkyl imidazoline quats of the mixture have at least one of R⁸ or R¹¹ which is a C₁₇ group or higher (17 or more carbons). Most frequently, both R⁸ and R¹¹ have chain lengths of C₁₇ or greater. Indeed, the most abundant dialkyl imidazoline quat molecule within the mixture will contain at least one C₂₁-C₂₂ alkyl group(depending upon whether it is R⁸ or R¹¹. In addition, the remaining R⁸ or R¹¹ group on such molecules would most likely include a C₂₁-C₂₂ alkyl group, a C₁₇-C₁₈ alkyl group or a C₁₉-C₂₀ alkyl group, most likely in that order of abundance.

The materials of this aspect of the present invention are preferred because they exhibit improved properties over other quat mixtures and mixtures containg relatively higher percentages of lower chain length alkyl groups, such as C₁₆ and under. Some of the quat mixtures of the present invention may have a lower degree of irritancy, better substantivity, better conditioning properties or generally enhanced performance.

Also, preferred in accordance with this aspect of the present invention are cosmetic and personal care products which include at least one active ingredient in an amount of between about 0.1 and 80%, more preferably between about 0.1 to about 40% by weight of the composition. The active ingredients may be a sunscreen, a pigment, a moisturizer, a film former, a detergent, a surfactant, a thickening agent, an emulsifier, an antiseptic agent, a conditioner or a deodorant. Where a surfactant is used, the amount of surfactant can range from between about 1% to about 75% by weight. In addition, the cosmetic or personal care product will include a mixture of dialkyl imidazoline quats having the general structure of formula II as described immediately above where either at least 50% of all are R⁸ and are R¹¹ groups have a chain length of C₁₇-C₃₅, and/or were at least 30% of a dialkyl imidazoline quats within the mixture of dialkyl imidazoline quats are di-substituted with a group having 19 carbons or greater as discussed above for R⁸ and R¹¹. The more preferred ranges described above in connection with the quat mixtures of this aspect of the invention are also preferred for use in the products described herein. This quat mixture may be provided in an amount of between about 0.1 and about 50% by weight of the product, more preferably between about 0.1 and 30%. However, in terms more conventionally used in the industry, these dialkyl imidazoline quats are provided such as that they have a cationic activity in the finished formulation of between about 0.1 and about 50%, more preferably, 0.1 and about 30%, and even more preferably about 0.1 and 20%. In a particularly preferred embodiment in accordance with the present invention, the cationic activity can be between 0.1% and about 10%.

In accordance with this aspect of the present invention, the cosmetic or personal care products can be a shampoo, a hair conditioner, a sunscreen formulation, a baby shampoo, baby bath product, hand dishwashing liquid, body wash, facial wash, bubble bath product, a hair coloring, agent, a hair styling or holding gel, cream, spray or liquid, or a cosmetic such as rouge, lipstick, eyeliner, mascara, foundation, blush and the like. Particularly preferred are personal care products selected from shampoos, conditioners, cream rinses or sunscreens and mixtures thereof. The cosmetic or personal care products can be aqueous or non-aqueous and may include up to about 98% of a solvent as previously described. In another preferred aspect, these products include at one hydrophobic ingredient which can be selected from the group consisting of botanical extracts, vitamin E, vitamin A, silicones, waxes and anti-oxidants. Additional ingredients can be added and these can include, for example, preservatives, fragrances, foam boosters, emollients, humectants and the like.

The compositions of the invention are further illustrated in the examples that follow.

### EXAMPLE 1

### Preparation of 1-methyl-1-((erucylamido-) ethyl)-2-erucyl immidazolinium methyl sulfate

3132 g (4.62 moles) of erucic acid and 216 g (2.1 moles) of diethylenetriamine are placed in a dry stirred pressure vessel fitted with a nitrogen inlet. The vessel is purged with nitrogen and heated to 170° C. for 4-5 hours. The reaction mixture is then heated to 180° C. and vacuum is applied for another 4-5 hours. The reaction mixture is cooled to 95-100° C. and approximately 1.5 kg of cetearyl alcohol is added. The reaction mixture is further cooled to 75-80° C. and 250 g of dimethyl sulfate is slowly added with stirring. Once all dimethyl sulfate is added, the reaction mixture is held at 75-80° C. for approximately one hour, providing 1-methyl-1-((erucylamido-)ethyl)-2-erucyl immidazolinium methyl sulfate as the product.

### EXAMPLE 2

### Preparation of 1-methyl-1-(erucic rapeseed-)-ethyl)-2-(erucic rapeseed-) immidazolinium methyl sulfate (Mixture of Dialkyl Imidazoline Quats of Hydrogenated Rapeseed Oil)

1843.6 g (1.88 moles) of hydrogenated rapeseed oil and 283.34 g (2.75 moles) of diethylenetriamine were placed in a dry stirred pressure vessel fitted with a nitrogen inlet. The vessel was purged with nitrogen and heated to 165° C. for 5 hours until a base value of 76 was reached. The reaction mixture was then heated to 190° C. and vacuum was applied for 5 hours to obtain a 94% tertiary amine content. The resulting imidazoline intermediate was then cooled to 95° C. and 1772 g of cetearyl alcohol were added to act as solvent. The reaction mixture was further cooled to 85° C. and 330 g (2.6 moles) of dimethyl sulfate were slowly added over a 30 minute period with stirring. Once all dimethyl sulfate was added, the reaction mixture was held at 85-90° C. for another 60 minutes. The resulting light yellow solid product included di-hydrogenated rapeseed oil imidazoline quat and cetearyl alcohol. The cationic activity of the mixture was 54%. The product was capable of being flaked or pastillated.

### EXAMPLE 3

Preparation of 1-methyl-1-N-(n-propyl)-2-erucyl immidazolinium methyl sulfate.

1790 g (2.64 moles) of erucid acid and 245 g (2.4 moles) of N-(n-propyl)-N-ethyleneamino)-diamine are placed in a dry stirred pressure vessel fitted with a nitrogen inlet. The vessel is purged with nitrogen and heated to 195° C. for 4-5 hours. The reaction mixture is then heated to 190° C. and vacuum is applied for another 4-5 hours. The reaction mixture is cooled to 100-105° C. and approximately 1.6 kg of cetearyl alcohol is added. The reaction mixture is further cooled to 75-80° C. and approximately 280 g of dimethyl sulfate is slowly added with stirring. Once all dimethyl sulfate is added, the reaction mixture is held at 85-90° C. for approximately one hour, providing 1-methyl-1-N-(n-propyl)-2-erucyl immidazolinium methyl sulfate as the product.

### EXAMPLE 4

### Quat Raw Material 1

Quat Raw Material 1 has the following composition:

| Function | Components |
|---|---|
| Quat(s) | Mixture M4 |
| | (Table 3) |
| Solvent | Mixture of |
| | cetearyl alcohol |
| | (80%) and 1,3- |
| | butanediol (20%) |

Cationic activity of the Quat Raw Material 1 is 45%.

### EXAMPLE 5

### Quat Raw Material 2

Quat Raw Material 2 has the following composition:

| Function | Component |
|---|---|
| Quat | Mixture of Table 7 |
| Solvent | Cetyl alcohol |

Cationic activity of the Quat Raw Material 2 is 25%.

### EXAMPLE 6

### Sunscreen Lotion

A sunscreen lotion includes the following ingredients:

Phase A

| Ingredient(s) | % W/W |
|---|---|
| Di-erucic | 1.0 |
| imidazoline quat | |
| Benzophenone 3 | 6.0 |
| Cetearyl Alcohol | 4.0 |
| Crodamol OS | 15.0 |
| (Octyl Stearate) | |
| Octyl | 7.5 |
| Methoxycinnamate | |

Phase B

| Ingredient | W/W % |
|---|---|
| Water | 65.50 |

Phase C

| Ingredient | W/W % |
|---|---|
| Germaben II | 1.0 |
| (preservative) | |

The sunscreen lotion is prepared as follows. The ingredients of Phase A are combined and heated to 75° C. In a separate vessel, the ingredients of Phase B are also combined and heated to 75° C. Phase A is added to Phase B with stirring; and the stirring is continued while the combined phases are cooled to 40° C. Phase C is added, the cooling is continued to 25° C., providing the desired lotion.

### EXAMPLE 7

### Sunscreen Spray Lotion

A sunscreen spray lotion includes the following ingredients:

Phase A

| Ingredient | % W/W |
|---|---|
| Di-erucic | 1.0 |
| imidazoline quat | |
| PPG-3 Benzyl | 11.0 |
| Myristate | |
| Benzophenone 3 | 6.0 |
| Octyl | 7.0 |
| Methoxycinnamate | |
| Menthyl | 5.0 |
| Anthranilate | |
| Cromollient SCE | 3.0 |
| (Di-PPG-2 Myreth- | |
| 10 Adipate) | |

Phase B

| Ingredient | W/W % |
|---|---|
| Water | 66 |
| Sodium Hydroxide | 0.1 |

Phase C

| Ingredient | W/W % |
|---|---|
| Germaben II | 1.0 |
| (preservative) | |

The sunscreen spray lotion is prepared as follows. The ingredients of Phase A are combined and heated to 75° C. In a separate vessel, the ingredients of Phase B are also combined and heated to 75° C. Phase A is added to Phase B with stirring, and the stirring is continued while the combined phases are cooled to 40° C. Phase C is added, the cooling is continued to 25° C., providing the desired lotion.

### EXAMPLE 8

### Hair Conditioner

A hair conditioner includes the following ingredients:

Phase A

| | % |
|---|---|
| Ingredient | W/W |
| Di-C₂₀₋₂₄ Imidazoline | 1.0 |
| Quat | |
| Cromollient SCE | 5.0 |
| (Di-PPG-2 Myreth-10 | |
| Adipate) | |
| Cetyl Alcohol | 4.0 |

Phase B

| Ingredient | W/W % |
|---|---|
| Water | 89 |

Phase C

| Ingredient | W/W % |
|---|---|
| Germaben II | 1.0 |
| (preservative) | |

The hair conditioner is prepared as follows. The ingredients of Phase A are combined and heated to 75° C. In a separate vessel, the ingredients of Phase B are also combined and heated to 7.5° C. Phase A is added to Phase B with stirring, and the stirring is continued while the combined phases are cooled to 40° C. Phase C is added, the cooling is continued to 25° C., providing the desired lotion.

### EXAMPLE 9

### Soft & Shine Conditioner

A soft and shine conditioner includes the following ingredients.

Phase A

| Ingredient | % W/W |
|---|---|
| Water | 86.26 |
| Mixture of Di-behenyl | 2.14 |
| imidazolinium Methosulfate | |
| and Cetrimonium Methasulfate | |
| (7/3 w/w) in Cetearyl alcohol | |
| (70% actives) | |
| CRODACOL C-70 (Cetyl | 1.00 |
| Alcohol) | |
| CRODACOL S-70 (Stearyl | 3.00 |
| Alcohol) | |
| CRILLET 3 (Polysorbate 60) | 1.00 |

Part B

| Ingredient | W/W % |
|---|---|
| INCROMINE SB | 0.5 |
| (Stearamidopropyl | |
| Dimethylamine) | |
| Cyclopentasiloxane (and) | 4.0 |
| Dimethicone (1) | |
| Dimethicone (2) | 0.5 |
| Disodium EDTA | 0.2 |
| Propylene Glycol (and) | 1.00 |
| Diazolidinyl Urea (and) | |
| Methylparaben | |
| (and) Propylparaben (3) | |
| Citric acid | 0.4 |

Part C

| Ingredient | W/W % |
|---|---|
| Germaben II | 1.0 |
| (Preservative) | |

### EXAMPLE 10

### Vitamin E-Containing Conditioner

The vitamin E-containing conditioner has the following ingredients.

Phase A

| Ingredient | % W/W |
|---|---|
| Deionized Water | 92.50 |
| CRODACOL S-70 (Stearyl Alcohol) | 3.80 |
| Mixture of Di-behenyl imidazolinium | 2.20 |
| Methosulfate and Cetrimonium | |
| Methasulfate (7/3 w/w) in Cetearyl | |
| alcohol (70% actives) | |

Part B

| Ingredient | W/W % |
|---|---|
| DL-α Tocopherol | 0.50 |
| Acetate (1) | |

Part C

| Ingredient | W/W % |
|---|---|
| Propylene Glycol (and) Diazolidinyl | 1.00 |
| Urea (and) Methylparaben | |
| (and) Propylparaben (2) | |

Preparation is as follows: combine Part A ingredients with mixing and heat to 75-80° C. Cool to 35° C. and add Parts B and C one at a time, mixing well.

### EXAMPLE 11

### Deposition of Vitamin E on Hair (Comparative Experiment 1)

Virgin and bleached hair tresses were treated with a simple conditioning formula containing 0.5% of the vitamin and 1.5% of either mixture of Di-behenyl imidazolinium Methosulfate and Cetrimonium Methosulfate (7/3 w/w) or Behentrimonium Chloride. The actives were present at the same level. Tresses were washed for 30 seconds and rinsed, after which the deposited Vitamin E was extracted with a solvent and measured by UV absorbance.

The results are shown below

Deposition of Vitamin E
(mg of Vit E/100 g of hair)

| Ingredient | Virgin Hair | Bleached Hair |
|---|---|---|
| Behentrimonium Chloride | 86.3 | 57.6 |
| Mixture of Di-behenyl | 180 | 80.9 |
| imidazolinium Methosulfate | | |
| and Cetrimonium | | |
| Methasulfate (7/3 w/w) | | |

### EXAMPLE 12

### Deposition of Vitamin E (Comparative Experiment 2)

Test conditioning shampoo formulations A and B were prepared using a mixture of Di-behenyl imidazolinium Methosulfate and Cetrimonium Methasulfate in 7/3 w/w ratio of quats to one another as the active conditioning ingredient. The added conditioning ingredient, which is generally derived from HEAR oil, contained 70% active quats by cationic activity in cetearyl alcohol. Test formulation A contained 1% of quat by cationic activity and test formulation A contained 0.5% of quat by cationic activity. Polyquaternium-10, a well-known polymeric conditioner, was used in the reference formulation C. The smaller amount of Polyquaternium-10 was used to reflect the cost benefit consideration.

Hair samples were treated with the respective conditioning shampoo for 3 minutes and rinsed off under 40° C. running tap water with a flow rate of 2.51/min for 20 seconds. The total substantivity was determined by two consecutive extractions by PVCS Method # 7-1. Only trace of Vitamin E was detected in the third extraction solution.

The determined total substantivity of Vitamin E delivered from these conditioning shampoo samples is presented below:

| | Average Substantivity | | |
|---|---|---|---|
| | (mg Vit E/100 g hair) | | |
| Ingredient//Amount | by PVCS Method # 7-1 | | |
| added to the Test | First | Second | |
| Shampoo | extraction | extraction | Total |
| Mixture of Di-behenyl | 52.7 | 23.1 | 75.8 |
| imidazolinium | | | |
| Methosulfate and | | | |
| Cetrimonium | | | |
| Methasulfate (7/3 w/w)// | | | |
| 1% cationic activity | | | |
| Mixture of di-behenyl | 46.4 | 44.6 | 91.0 |
| imidazolinium | | | |
| methosulfate and | | | |
| cetrimonium | | | |
| methasulfate (7/3 w/w | | | |
| ratio) in cetearyl | | | |
| alcohol (70% | | | |
| actives)// | | | |
| 0.5% cationic activity | | | |
| Polyquaternium-10// | 24.3 | 15.6 | 39.9 |
| 0.3% by cationic | | | |
| activity | | | |

The conditioning shampoo samples containing dibehenyl imidazolinium methosulfate showed better deposition of Vitamin E onto hair surface than the sample containing Polyquaternium-10. Also, the deposition of Vitamin E on hair surface was enhanced by an increase in the concentration of di-behenyl imidazolinium methosulfate in the formulation.

Unless stated to the contrary, any use of the words such as “including,” “containing,” “comprising,” “having” and the like, means “including without limitation” and shall not be construed to limit any general statement that it follows to the specific or similar items or matters immediately following it. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A mixed composition comprising: a mixture of dialkyl imidazoline quats of the formula (II): 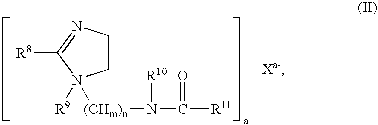
wherein X is a salt-forming anion;
a is the ionic charge of X;
n is 1, 2 or 3;
m is 1 or 2;
R⁹ is a C₁-C₆ alkyl group or a benzyl group, either of which may be substituted or unsubstituted;
R¹⁰ is hydrogen or a C₁-C₆ alkyl group which may saturated or unsaturated and may be hydroxy substituted; and
R⁸ and R¹¹ may be the same or different and are alkyl, alkylhydroxy, alkyl amido, alkylaryl amido or alkylhydroxy amido group wherein at least about 30% of said R⁸ and R¹¹ are alkyl groups of at least 19 carbons in length which may be straight, branched or cyclized, may be saturated or unsaturated and may be hydroxy substituted or unsubstituted.

2. The mixed composition of claim 1, wherein said at least 30% of said R⁸ and R¹¹ are C₁₉-C₂₉ alkyl groups which may be straight, branched or cyclized, may be saturated or unsaturated and may be hydroxy substituted or unsubstituted.

3. The mixed composition of claim 2, wherein said at least 30% of said R⁸ and R¹¹ are C₁₉-C₂₆ alkyl groups which may be straight, branched or cyclized, may be saturated or unsaturated and may be hydroxy substituted or unsubstituted.

4. The mixed composition of claim 2, wherein said at least about 40% of said R⁸ and R¹¹ are C₁₉-C₂₉ alkyl groups which may be straight, branched or cyclized, may be saturated or unsaturated and may be hydroxy substituted or unsubstituted.

5. The mixed composition of claim 4, wherein said at least about 50% of said R⁸ and R¹¹ are C₁₉-C₂₉ alkyl groups which may be straight, branched or cyclized, may be saturated or unsaturated and may be hydroxy substituted or unsubstituted.

6. A cosmetic or personal care product comprising:
at least one active ingredient in an amount of between about 0.1 and 80% by weight of said product, said active ingredient being a sunscreen, a pigment, a moisturizer, a film former, a detergent, a surfactant, a thickening agent, an emulsifier, an antiseptic agent, a conditioner or a deodorant;
a mixture including at least two dialkyl imidazoline quats said dialkyl imidazoline quats having a structure of the formula (II): 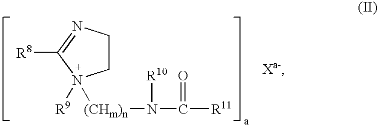
wherein X is a salt-forming anion;
a is the ionic charge of X;
n is 1, 2 or 3;
m is 1 or 2;
R⁹ is a C₁-C₆ alkyl group or a benzyl group, either of which may be substituted or unsubstituted;
R¹⁰ is hydrogen or a C₁-C₆ alkyl group which may saturated or unsaturated and may be hydroxy substituted; and
R⁸ and R¹¹ may be the same or different and are alkyl, alkylhydroxy, alkyl amido, alkylaryl amido or alkylhydroxy amido group wherein at least about 30% of said R⁸ and R¹¹ are alkyl groups of at least 19 carbons in length which may be straight, branched or cyclized, may be saturated or unsaturated and may be hydroxy substituted or unsubstituted.

7. The cosmetic or personal care product of claim 6, wherein said at least about 40% of said dialkyl imidazoline quats both R⁸ and R¹¹ are C₁₉-C₂₉ alkyl groups which may be straight, branched or cyclized, may be saturated or unsaturated and may be hydroxy substituted or unsubstituted.

8. The cosmetic or personal care product as in claim 6 or 7, wherein said mixture of at least two dialkyl imidazoline quats is provided in an amount such that said quat mixture has a cationic activity in the product of between about 0.5 to about 20%.

9. The cosmetic or personal care product of claim 8, wherein said mixture of at least two dialkyl imidazoline quats is provided in an amount such that said quat mixture has a cationic activity in the product of between about 0.5 to about 10%.

10. The cosmetic or personal care product as in claim 6 or 7 further comprising up to about 98% of a solvent.

11. The cosmetic or personal care product as in claim 10 wherein said solvent includes water.

12. The cosmetic or personal care product as in claim 6 or 7 wherein said product is a personal care product selected from a shampoo, conditioner, cream rinse or sunscreen and mixtures thereof.

13. A mixed composition comprising: a mixture of dialkyl imidazoline quats of the formula (II): 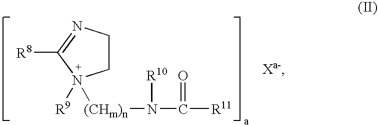
wherein X is a salt-forming anion;
a is the ionic charge of X;
n is 1, 2 or 3;
m is 1 or 2;
R⁹ is a C₁-C₆ alkyl group or a benzyl group, either of which may be substituted or unsubstituted;
R¹⁰ is hydrogen or a C₁-C₆ alkyl group which may saturated or unsaturated and may be hydroxy substituted; and
R⁸ and R¹¹ may be at the same or different and are alkyl, alkylhydroxy, alkyl amido, alkylaryl amido or alkylhydroxy amido group wherein at least about 30% of said R⁸ and R¹¹ are alkyl groups of at least 21 carbons in length which may be straight, branched or cyclized, may be saturated or unsaturated and may be hydroxy substituted or unsubstituted.

14. The mixed composition of claim 13, wherein said at least 30% of said R⁸ and R¹¹ are C₂₁-C₂₉ alkyl groups which may be straight, branched or cyclized, may be saturated or unsaturated an may be hydroxy substituted or unsubstituted.

15. The mixed composition of claim 14, wherein said at least 30% of said R⁸ and R¹¹ are C₂₁-C₂₂ alkyl groups which may be straight, branched or cyclized, may be saturated or unsaturated and may be hydroxy substituted or unsubstituted.

16. The mixed composition of claim 15, wherein said at least about 30% of said R⁸ and R¹¹ are C₂₁-C₂₂ alkyl groups which may be straight, branched or cyclized, may be saturated or unsaturated and may be hydroxy substituted or unsubstituted.

17. The mixed composition of claim 14, wherein said at least about 40% of said R⁸ and R¹¹ are C₁₉-C₂₉ alkyl groups which may be straight, branched or cyclized, may be saturated or unsaturated and may be hydroxy substituted or unsubstituted.

18. A cosmetic or personal care product comprising:
at least one active ingredient in an amount of between about 0.1 to 80% by weight of said product, said active ingredient being a sunscreen, a pigment, a moisturizer, a film former, a detergent, a surfactant, a thickening agent, an emulsifier, an antiseptic agent, a conditioner or a deodorant;
a mixture including at least two dialkyl imidazoline quats of said dialkyl imidazoline quats having a structure of the formula: 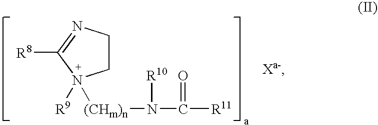
wherein X is a salt-forming anion;
a is the ionic charge of X;
n is 1, 2 or 3;
m is 1 or 2;
R⁹ is a C₁-C₆ alkyl group or a benzyl group, either of which may be substituted or unsubstituted;
R¹⁰ is hydrogen or a C₁-C₆ alkyl group which may saturated or unsaturated and may be hydroxy substituted; and
R⁸ and R¹¹ may be the same or different and are alkyl, alkylhydroxy, alkyl amido, alkylaryl amido or alkylhydroxy amido group wherein at least about 30% of said R⁸ and R¹¹ are alkyl groups of at least 21 carbons in length which may be straight, branched or cyclized, may be saturated or unsaturated and may be hydroxy substituted or unsubstituted.

19. The cosmetic or personal care product of claim 18, wherein said at least about 30% of said R⁸ and R¹¹ are C₁₉-C₂₉ alkyl groups which may be straight, branched or cyclized, may be saturated or unsaturated and may be hydroxy substituted or unsubstituted.

20. The cosmetic or personal care product as in claim 18 or 19, wherein said mixture of at least two dialkyl imidazoline quats is provided in an amount such that said quat mixture has a cationic activity in the product of between about 0.5 to about 20%.

21. The cosmetic or personal care product of claim 20, wherein said mixture of at least two dialkyl imidazoline quats is provided in an amount such that said quat mixture has a cationic activity in the product of between about 0.5 to about 10%.

22. The cosmetic or personal care product as in claim 18 or 19 further comprising up to about 98% of a solvent.

23. The cosmetic or personal care product as in claim 22, wherein said solvent includes water.

24. The cosmetic or personal care product as in claim 18 or 19, wherein said product is a personal care product selected from a shampoo, conditioner, cream rinse or sunscreen and mixtures thereof.

25. The cosmetic or personal care product as in any one of claims 1, 6, 13, and 18, wherein said salt-forming anion is selected from the group consisting of chloride, bromide, iodide, fluoride, sulfate, methyl sulfate, methanebenzylsulfonate, phosphate, nitrite, nitrate, carboxylate, and mixtures thereof.
